(19) United States
(12) Patent Application Publication
(10) Pub. No.: US 2005/0238711 A1
(43) Pub. Date: Oct. 27, 2005

(54) Filled depression tablets and method for their production II

(76) Inventors: Alexander Lambotte, Duesseldorf (DE); Thomas Holderbaum, Monheim (DE); Maren Jekel, Barcelona (ES)

Correspondence Address: HENKEL CORPORATION, THE TRIAD, SUITE 200, 2200 RENAISSANCE BLVD., GULPH MILLS, PA, 19406, US

(21) Appl. No.: 11/130,067

(22) Filed: May 16, 2005

Related U.S. Application Data
(63) Continuation of application No. PCT/EP03/12324, filed on Nov. 5, 2003.

(30) Foreign Application Priority Data
Nov. 16, 2002 (DE) 102 53 479.9

(51) Int. Cl. A61K 9/20

(52) U.S. Cl. 424/464

(57) ABSTRACT

A tablet having a base tablet having a depression and a solid filling the depression, wherein the solid filling the depression is secured in the depression by a fixative, and wherein a space is formed between the depression surface and the solid that is not filled with the fixative.

### CROSS REFERENCE TO RELATED APPLICATIONS

This application is a continuation under 35 U.S.C. § 365(c) and 35 U.S.C. § 120 of international application PCT/EP2003/012324, filed on Nov. 5, 2003. This application also claims priority under 35 U.S.C. § 119 of DE 102 53 479.9, filed Nov. 16, 2002, which is incorporated herein by reference in its entirety.

### BACKGROUND OF THE INVENTION

The present invention relates to depression tablets which have a filling and to processes for their production. Such depression tablets find use in a wide variety of fields, for example pharmacy, crop protection, and the food or animal feed industry. In particular, the present invention relates, however, to filled depression tablets which comprise washing or cleaning constituents and find use as laundry detergent or cleaning composition tablets.

Filled depression tablets may be produced, for example, according to the teaching of EP 979 864 A1l, by compressing a first premixture to form a depression tablet, introducing a further premixture into the depression and compressing it to form a core.

One alternative consists in separately prefabricating depression tablet and core, and adhesive-bonding the core into the depression. This method is complicated in apparatus terms, since the core has to be placed exactly. In the case of an inexactly placed core, it can break on insertion or damage the depression tablet. In addition, the visual appearance is impaired when the core is not positioned exactly in the middle of the depression.

It is an object of the present invention to provide filled depression tablets which can be produced without damage either in the core or in the depression and have a visually appealing appearance. At the same time, good storage stability should be achieved even for tablets which comprise mutually incompatible ingredients. In addition, a production process should be provided which enables simple and inexpensive production of large numbers of items.

### DESCRIPTION OF THE INVENTION

In a first embodiment, the present invention therefore provides filled depression tablets comprising a base tablet which has a depression and a solid depression filling, characterized in that the depression filling is secured in the depression with the aid of a fixative, and a fixative-unfilled space is formed between the bottom of the depression in the base tablet and the depression filling.

In contrast to the depression tablets known from the prior art, the core is not adhesive-bonded into the depression with the aid of a fixative which is disposed between depression bottom and core. Rather, the securing is effected with the aid of a fixative which does not fully cover the bottom of the depression and accordingly forms a fixative-unfilled space between the underside of the core and the depression bottom.

The inventive depression tablets comprise a base tablet (also referred to below as base tablet) which has a depression. Such depression tablets may, depending on the shape of the tablet and of the depression, assume a wide variety of shapes.

The inventive tablets may assume any geometric shape, preference being given in particular to concave, convex, biconcave, biconvex, cubic, tetragonal, orthorhombic, cylindrical, spherical, cylinder segment-like, disk-shaped, tetrahedral, dodecahedral, octahedral, conical, pyramidal, ellipsoidal, pentagonally, heptagonally and octagonally prismatic, and rhombohedral shapes. It is also possible to realize entirely irregular outlines such as arrow or animal shapes, trees, clouds, etc. When the inventive tablets have corners and edges, these are preferably rounded off. As an additional visual differentiation, preference is given to an embodiment with rounded corners and beveled (chamfered) edges.

The inventive tablets can of course also be produced in multiphase form. For reasons of process economics, two-layer tablets have been found to be particularly useful here.

The shape of the cavity may also be freely selected within wide limits. Thus, the depressions of the inventive tablets may assume any geometric shape, preference being given in particular to concave, convex, biconcave, biconvex, cubic, tetragonal, orthorhombic, cylindrical, spherical, cylinder segment-like, disk-shaped, tetrahedral, dodecahedral, octahedral, conical, pyramidal, ellipsoidal, pentagonally, heptagonally and octagonally prismatic and rhombohedral shapes. It is also possible to realize entirely irregular depression outlines such as arrow or animal shapes, trees, clouds, etc. When the depression boundary has corners and edges, these are preferably rounded off. As an additional visual differentiation, preference is given to an embodiment with rounded corners and beveled (chamfered) edges.

The size of the depression in comparison to the entire tablet depends upon the desired end use of the tablets. Irrespective of the end use, preference is given to laundry detergents and cleaning composition tablets in which the volume ratio of tablet to cavity is from 2:1 to 100:1, preferably from 3:1 to 80:1, more preferably from 4:1 to 50:1 and in particular from 5:1 to 30:1. The volume ratio is calculated from the volume of the inventive finished tablet, i.e. of the tablet which has the cavity and the fill volume of the cavity. The difference in the two volumes gives the volume of the tablet with cavity. In other words: when the tablet has, for example, an orthorhombic shape having the side lengths 2, 3 and 4 cm and has a depression having a volume of 2 cm³, the volume of this “base tablet” is 22 cm³. In this example, the ratio of the volumes is thus 11:1. At tablet:cavity volume ratios below 2:1, which can of course also be realized in accordance with the invention, the instability of the walls can rise.

Similar statements can be made with regard to the surface area fractions which are made up by the tablet with the cavity (“base tablet”) or the opening surface area of the cavity in the total surface area of the tablet. Preference is given here to laundry detergent and cleaning composition tablets in which the surface area of the opening(s) of the cavity/cavities make(s) up from 1 to 25%, preferably from 2 to 20%, more preferably from 3 to 15% and in particular from 4 to 10%, of the total surface area of the tablet. The total surface area of the tablet corresponds here again to the total surface area of the tablet with “hypothetically closed” cavity, i.e., in the above example, regardless of the opening surface area of the depression, 52 cm². In the case of such an exemplary tablet, in preferred embodiments of the present invention, the opening(s) of the cavity thus has/have a surface area of from 0.52 to 13 cm², preferably from 1.04 to 10.4 cm², more preferably from 1.56 to 7.8 cm² and in particular from 2.08 to 5.2 cm².

The bottom of the depression in the depression tablet may be planar. Planar depression bottoms may run entirely parallel to the encumbent surface of the tablet, but may also be inclined. However, it is also possible to provide a nonplanar depression bottom, for example a concave bottom. This is preferred, since the production of the corresponding depression tablets is better. A compression punch with protruding spike, which is required to produce depression tablets, better displaces the particulate premixture on compression when the spike is not entirely plane-parallel to the compression surface, but rather enables premixture particles to slide away from the pressure zone to the later webs of the depression.

Being the depression filling, the core can virtually fully fill the opening surface area of the depression. However, it is also possible that the core has a distinctly smaller diameter than the opening diameter of the depression. In the context of the present invention, it is preferred that the maximum horizontal cross-sectional surface area of the core makes up from 0.2 to 0.98 times the opening surface area of the depression, preferably from 0.5 to 0.95 times the opening surface area of the depression and in particular from 0.7 to 0.9 times the opening surface area of the depression.

The fixative may secure the depression filling to the depression tablet in various ways. In cases in which the depression filling does not fully cover the depression bottom, the fixative may fill the gap between the depression walls and the edges of the core filling partly, i.e. in the edge region of the underside of the core, and thus secure it in the depression. Such inventive depression tablets in which a contact surface is formed between the bottom of the depression in the base tablet and the fixative are preferred in accordance with the invention. In this case, the fixative, according to the invention, must not completely fill the intermediate space between the underside of the core and the depression bottom; rather, a fixative-unfilled space, i.e. a hollow area, has to be formed. The fixative may also bond the core to the edge of the depression via its edge. In addition, the fixative may cover the upper side of the depression filling and thus ensure securing—the latter way is also possible when the depression filling fully covers the depression bottom, i.e. the maximum horizontal cross section of the core is smaller than the opening area of the depression.

Preferred inventive depression tablets are characterized in that the fixative fully covers the upper side of the depression filling.

It is preferred in the context of the present invention when the depression filling does not fully cover the depression bottom. In addition, it is preferred that the depression filling does not touch the side walls of the depression, but rather is spaced apart from them. Preference is given here to inventive depression tablets in which the distance between the lateral boundary walls of the depression and the depression filling is from 0.5 to 10 mm, preferably from 1 to 9 mm, more preferably from 2 to 8 mm and in particular from 3 to 7 mm.

As a further constituent, the inventive depression tablets comprise a solid depression filling. This filling, also referred to as the “core”, is a tablet which can be produced, for example, by casting, sintering, extrusion, calendering, etc. However, it is preferred that the depression filling is a tablet.

Preference is given here to inventive depression tablets in which the depression filling is a tablet which has a density of ≧1 gcm⁻³, preferably ≧1.25 gcm⁻³ and in particular ≧1.5 gcm⁻³.

The depression filling is secured in the depression with the aid of a fixative. Useful fixatives include a wide variety of substances. Preference is given in the context of the present invention to solutions, suspensions, dispersions or emulsions of adhesive substances in volatile solvents.

Particular preference is given to using meltable substances as the fixative. Preference is given here to inventive depression tablets, characterized in that the fixative comprises one or more meltable substance(s) which has/have a melting point between 30 and 250° C., preferably between 35 and 200° C. and in particular between 40 and 180° C.

Some examples of fixatives which are suitable in the context of the present invention and have the criterion of a melting point between 30 and 250° C., are summarized in the following table:

| | Melting | |
|---|---|---|
| | point | Solubility |
| | [° C.] | [g/l H₂O] |
| ammonium aluminum sulfate dodecahydrate | 93 | 150 |
| potassium aluminum sulfate dodecahydrate | 92 | 110 |
| aluminum sulfate monohydrate | 90 | 600 |
| aluminum sulfate octadecahydrate | 90 | 600 |
| sodium phosphinate monohydrate | 90 | 1000 |
| sodium dihydrogenphosphate | 100 | 1103 |
| sodium dihydrogenphosphate monohydrate | 100 | 1103 |
| sodium ammonium hydrogenphosphate tetrahydrate | 79 | 167 |
| disodium hydrogenphosphate heptahydrate | 48 | 154 |
| trisodium phosphate dodecahydrate | 75 | 258 |
| tripotassium phosphate heptahydrate | 46 | 900 |
| ammonium iron(II) sulfate hexahydrate | 100 | 269 |
| iron sulfate heptahydrate | 64 | 400 |
| glucose | 83 | 820 |
| magnesium acetate tetrahydrate | 80 | 1200 |
| manganese(II) chloride tetrahydrate | 58 | 1980 |
| sodium acetate trihydrate | 58 | 762 |
| sodium hydrogensulfate monohydrate | 58 | 670 |
| sodium carbonate peroxohydrate | 60 | 150 |
| sodium thiosulfate pentahydrate | 48 | 680 |
| potassium sodium tartrate tetrahydrate | 70-80 | 630 |
| D-(+)-glucose monohydrate | 83 | 820 |
| zinc acetate dihydrate | 100 | 430 |
| zinc sulfate heptahydrate | 40 | 960 |

In the context of the present invention, particularly suitable fixatives have been found to be the sugars, sugar acids and sugar alcohols. These substances are generally not only sufficiently soluble but also additionally feature low costs and good processibility. For instance, sugars and sugar derivatives, especially the mono- and disaccharides and derivatives thereof, can be processed, for example, in the form of their melts, these melts having good dissolution capability both for dyes and for many washing and cleaning substances. The solid bodies resulting from the solidification of the sugar melts additionally feature a smooth surface and advantageous appearance, such as high surface brightness or transparent appearance.

Preferred depression tablets in the context of the present invention are accordingly characterized in that the fixative is selected from the group of the sugars and/or sugar acids and/or sugar alcohols, preferably from the group of the sugars, more preferably from the group of the oligosaccharides, oligosaccharide derivatives, monosaccharides, disaccharides, monosaccharide derivatives and disaccharide derivatives and mixtures thereof, in particular from the group of glucose and/or fructose and/or ribose and/or maltose and/or lactose and/or sucrose and/or maltodextrin and/or Isomalt®.

The group of the sugars preferred as the fixative in the context of the present application includes, from the group of the mono- and disaccharides and derivatives of mono- and disaccharides, especially glucose, fructose, ribose, maltose, lactose, sucrose, maltodextrin and Isomalt®, and also mixtures of two, three, four or more mono- and/or disaccharides and/or the derivatives of mono- and/or disaccharides. For instance, particularly preferred fixatives are mixtures of Isomalt® and glucose, Isomalt® and lactose, Isomalt® and fructose, Isomalt® and ribose, Isomalt® and maltose, glucose and sucrose, Isomalt® and maltodextrin or Isomalt® and sucrose. The proportion by weight of Isomalt® in the total weight of the aforementioned mixtures is preferably at least 20% by weight, more preferably at least 40% by weight and in particular at least 80% by weight.

Also particularly preferred as fixatives are mixtures of maltodextrin and glucose, maltodextrin and lactose, maltodextrin and fructose, maltodextrin and ribose, maltodextrin and maltose or maltodextrin and sucrose. The proportion by weight of maltodextrin in the total weight of the aforementioned mixtures is preferably at least 20% by weight, more preferably at least 40% by weight and in particular at least 80% by weight.

In the context of the present application, maltodextrin refers to water-soluble carbohydrates obtained by enzymatic degradation of starch (dextrose equivalents, DE 3-20) having a chain length of 5-10 anhydroglucose units and a high proportion of maltose. Maltodextrins are added to foods to improve the rheological and calorific properties, only have a slight sweet taste and do not tend to retrograde. Commercial products, for example from Cerestar, are generally available as spray-dried, free-flowing powders and have a water content of from 3 to 5% by weight.

In the context of the present application, Isomalt® refers to a mixture of 6-O-α-D-glucopyranosyl-D-sorbitol (1,6-GPS) and 1-O-α-D-glucopyranosyl-D-mannitol (1,1-GPM). In a preferred embodiment, the proportion by weight of 1,6-GPS in the total weight of the mixture is less than 57% by weight. Such mixtures can be produced industrially, for example, by enzymatic rearrangement of sucrose to isomaltose and subsequent catalytic hydrogenation of the resulting isomaltose to form an odorless, colorless and crystalline solid.

Fixatives used with particular preference in the context of the present application are also the sugar acids. Sugar acids can be used advantageously as a constituent of the active phase alone or in combination with other substances, for example the abovementioned sugars, and particularly preferred sugar acids are from the group of gluconic acid, galactonic acid, mannonic acid, fructonic acid, arabinonic acid, xylonic acid, ribonic acid, 2-deoxyribonic acid. Particularly preferred fixatives also contain Isomalt® in addition to the sugar acids mentioned. The proportion by weight of Isomalt® in the total weight of these mixtures is preferably at least 20% by weight, more preferably at least 40% by weight and in particular at least 80% by weight, and particular preference is given to mixtures of Isomalt® with gluconic acid, Isomalt® with galactonic acid, Isomalt® with mannonic acid, Isomalt® with fructonic acid, Isomalt® with arabinonic acid, Isomalt® with xylonic acid, Isomalt® with ribonic acid and Isomalt® with 2-deoxyribonic acid.

A third group of advantageously usable fixatives is that of the sugar alcohols, of which preference is given in the context of the present application in particular to mannitol, sorbitol, xylitol, dulcitol and arabitol. The sugar alcohols may be used alone or as mixtures with one another or as a mixture with further sugars, sugar derivatives, sugar acids or sugar acid derivatives. Particular preference is given to using mixtures of sugar alcohols with Isomalt®, and particular preference is given to mixtures of Isomalt® with mannitol, Isomalt® with sorbitol, Isomalt® with xylitol, Isomalt® with dulcitol and Isomalt® with arabitol. The proportion by weight of Isomalt® in the total weight of these mixtures is preferably at least 20% by weight, more preferably at least 40% by weight and in particular at least 80% by weight.

When the fixatives used are the abovementioned sugars or sugar substances, it should be ensured with regard to the preferred use of the inventive products as laundry detergent or cleaning composition tablets that inadvertent consumption of the product by humans (for example children) is reliably prevented. Suitable substances for this purpose are bitter substances, i.e. compounds with a highly bitter taste, which, apart from the rise, caused by the taste and induced by the reflexes, in the secretion of the digestive glands and the spontaneous refusal to incorporate the bitter product, do not have any further pharmacological effects.

Bitter substances occur in a large number of plant families and are nonuniform in their chemical structure, but frequently have lactone or —CO—CH═CH— moieties. Of particular significance are the bitter substances with the secoiridoid structure, which are found preferentially in plants of the Gentianaceae and Menyanthaceae families, with the sesquiterpene basic structure, common in particular in species of the Asteraceae family, with a diterpene basic structure (Lamiaceae) and with the nortriterpene structure (quassinoids of the Simaroubaceae, limonoids of the Rutaceae and cucurbitacins of the Cucurbitaceae).

The filled depression tablets preferred in accordance with the invention comprise a bitter substance, particularly preferred depression tablets comprising the Bitrex® additive. Bitrex® (denatonium benzoate, [benzyldiethyl(2,6-xylylcarbamoyl)methylammonium benzoate], CAS No. 3734-33-6, MW 446.5, solubility (H₂O, 20° C.) 45 g/l, m.p. 163-170° C.) is a selective bitter substance and, by virtue of its extremely bitter taste, reliably prevents the consumption of the inventive products by humans.

The bitter substances are typically added in concentrations of from 0.0001 to 0.01% by weight, based in each case on the product. Preferred inventive tablets contain, for example, 0.0010% Bitrex.

The bitter substance is preferably used as a constituent of the fixative melt or as a coating over the entire tablet. Particular preference is given to spraying a solution or dispersion of the bitter substance onto the solidified fixative melt and to allowing the solvent to evaporate, which can be supported if appropriate by blowing with air, infrared heating or hot air. Particularly suitable for this process alternative are aqueous Bitrex® solutions.

An inventive tablet comprises basically two regions in which different ingredients may be present or different release mechanisms and dissolution kinetics can be realized: the depression tablet and the depression filling. In addition, suitable ingredients, for example dyes and/or fragrances, may also be incorporated in the fixative.

A suitable formulation of tablet and film material allows the time at which the substance present in the cavity is released to be predetermined. For example, the fixative may be soluble virtually instantly, so that the active substance present in the depression is metered into the washing or cleaning liquor right at the start of the washing or cleaning cycle. Alternatively, the fixative may be so sparingly soluble that the tablet is dissolved first and this releases the active substance present in the cavity.

Depending on this release mechanism, it is possible, for example, to realize tablets in which the depression filling is present dissolved in the cleaning liquor before the constituents of the tablet have dissolved, or after it has happened.

There now follows a description of the ingredients of the base tablet, which may at the same time be an ingredient of the depression filling, and a list of preferred physical parameters for base tablets and depression filling. Incorporation of particular constituents firstly allows the solubility of the filling of the cavity to be accelerated in a controlled manner, and the release of certain ingredients from this filling can secondly lead to advantages in the washing or cleaning process. Ingredients which are preferably localized at least in part in the depression filling are, for example, the surfactants, enzymes, soil release polymers, builders, bleaches, bleach activators, bleach catalysts, optical brighteners, silver protectants, etc., which are described below.

In preferred embodiments of the present invention, the base tablet has a high specific density. Preference is given in accordance with the invention to laundry detergent and cleaning composition tablets which are characterized in that the base tablet has a density above 1000 kgdm⁻³, preferably above 1025 kgdm⁻³, more preferably above 1050 kgdm⁻³ and in particular above 1100 kgdm⁻³.

Further details of physical parameters of the base tablet and of the finished laundry detergent and cleaning composition tablets and information on the production can be found below. There follows an illustration of the preferred ingredients of the base tablet.

Laundry detergent and cleaning composition tablets which are preferred in the context of the present invention are characterized in that the base tablet contains builders in amounts of from 1 to 100% by weight, preferably from 5 to 95% by weight, more preferably from 10 to 90% by weight and in particular from 20 to 85% by weight, based in each case on the weight of the base tablet.

The inventive laundry detergent and cleaning composition tablets may comprise all builders used customarily in laundry detergents and cleaning compositions, i.e., in particular zeolites, silicates, carbonates, organic cobuilders and, where no ecological objections to their use exist, also the phosphates.

Suitable crystalline, sheet-type sodium silicates have the general formula NaMSiₓO₂ₓ₊₁.H₂O where M is sodium or hydrogen, x is a number from 1.9 to 4, y is a number from 0 to 20, and preferred values for x are 2, 3 or 4. Preferred crystalline sheet silicates of the formula specified are those in which M is sodium and x assumes the values of 2 or 3. In particular, preference is given to both β- and also δ-sodium disilicates Na₂Si₂O₅.yH₂O.

It is also possible to use amorphous sodium silicates having an Na₂O:SiO₂ modulus of from 1:2 to 1:3.3, preferably from 1:2 to 1:2.8 and in particular from 1:2 to 1:2.6, which have retarded dissolution and secondary washing properties. The retardation of dissolution relative to conventional amorphous sodium silicates may have been brought about in a variety of ways, for example by surface treatment, compounding, compacting or by overdrying. In the context of this invention, the term “amorphous” also includes “X-ray-amorphous”. This means that, in X-ray diffraction experiments, the silicates do not afford any sharp X-ray reflections typical of crystalline substances, but rather yield at best one or more maxima of the scattered X-radiation, which have a width of several degree units of the diffraction angle. However, it may quite possibly lead to even particularly good builder properties if the silicate particles in electron diffraction experiments yield vague or even sharp diffraction maxima. This is to be interpreted such that the products have microcrystalline regions with a size of from 10 to several hundred nm, preference being given to values up to a maximum of 50 nm and in particular up to a maximum of 20 nm. Such X-ray-amorphous silicates likewise have retarded dissolution compared with conventional waterglasses. Special preference is given to compacted amorphous silicates, compounded amorphous silicates and overdried X-ray-amorphous silicates.

Laundry detergent and cleaning composition tablets which are preferred in the context of the present invention are characterized in that the base tablet comprises silicate(s), preferably alkali metal silicates, more preferably crystalline or amorphous alkali metal disilicates, in amounts of from 10 to 60% by weight, preferably from 15 to 50% by weight and in particular from 20 to 40% by weight, based in each case on the weight of the base tablet.

The finely crystalline, synthetic, bound water-containing zeolite used is preferably zeolite A and/or P. The zeolite P is more preferably Zeolite MAP® (commercial product from Crosfield). Also suitable, however, are zeolite X, and mixtures of A, X and/or P. Also commercially available and usable with preference in accordance with the invention is, for example, a cocrystal of zeolite X and zeolite A (approx. 80% by weight of zeolite X), which is sold by CONDEA Augusta S.p.A. under the trade name VEGOBOND AX® and can be described by the formula
nNa₂O.(1-n)K₂O.Al₂O₃.(2-2.5)SiO₂.(3.5-5.5)H₂O.

The zeolite may be used either as a builder in a granular compound or in a kind of “powdering” of the entire mixture to be compacted, and both ways of incorporating the zeolite into the premixture are typically utilized. Suitable zeolites have an average particle size of less than 10 μm (volume distribution; measurement method: Coulter Counter) and preferably contain from 18 to 22% by weight, in particular from 20 to 22% by weight, of bound water.

It is of course also possible to use the commonly known phosphates as builder substances, as long as such a use is not to be avoided for ecological reasons. Among the multitude of commercially available phosphates, the alkali metal phosphates, with particular preference for pentasodium triphosphate or pentapotassium triphosphate (sodium tripolyphosphate or potassium tripolyphosphate), have the greatest significance in the laundry detergents and cleaning products industry.

Alkali metal phosphates is the collective term for the alkali metal (especially sodium and potassium) salts of the various phosphoric acids, for which a distinction may be drawn between metaphosphoric acids (HPO₃)ₙ and orthophosphoric acid H₃PO₄, in addition to higher molecular weight representatives. The phosphates combine a number of advantages: they act as alkali carriers, prevent limescale deposits on machine components and lime encrustations in fabrics, and additionally contribute to the cleaning performance.

Sodium dihydrogenphosphate, NaH₂PO₄, exists as the dihydrate (density 1.91 gcm⁻³, melting point 60°) and as the monohydrate (density 2.04 gcm⁻³). Both salts are white powders which are very readily soluble in water and which lose the water of crystallization upon heating and are converted at 200° C. to the weakly acidic diphosphate (disodium hydrogendiphosphate, Na₂H₂P₂O₇), and at higher temperature to sodium trimetaphosphate (Na₃P₃O₉) and Maddrell's salt (see below). NaH₂PO₄ reacts acidically; it is formed when phosphoric acid is adjusted to a pH of 4.5 using sodium hydroxide solution and the slurry is sprayed. Potassium dihydrogenphosphate (primary or monobasic potassium phosphate, potassium biphosphate, KDP), KH₂PO₄, is a white salt of density of 2.33 gcm⁻³, has a melting point of 253° [decomposition with formation of potassium polyphosphate (KPO₃)ₓ] and is readily soluble in water.

Disodium hydrogenphosphate (secondary sodium phosphate), Na₂HPO₄, is a colorless crystalline salt which is very readily soluble in water. It exists in anhydrous form and with 2 mol of water (density 2.066 gcm⁻³, loss of water at 95°), 7 mol of water (density 1.68 gcm⁻³, melting point 48° with loss of 5 H₂O) and 12 mol of water (density 1.52 gcm⁻³, melting point 35° with loss of 5 H₂O), becomes anhydrous at 100° and, when heated more strongly, is converted to the diphosphate Na₄P₂O₇. Disodium hydrogenphosphate is prepared by neutralizing phosphoric acid with sodium carbonate solution using phenolphthalein as an indicator. Dipotassium hydrogenphosphate (secondary or dibasic potassium phosphate), K₂HPO₄, is an amorphous white salt which is readily soluble in water.

Trisodium phosphate, tertiary sodium phosphate, Na₃PO₄, are colorless crystals which have a density of 1.62 gcm⁻³ and a melting point of 73-76° C. (decomposition) in the form of the dodecahydrate, have a melting point of 100° C. in the form of the decahydrate (corresponding to 19-20% P₂O₅), and have a density of 2.536 gcm⁻³ in anhydrous form. (corresponding to 39-40% P₂O₅). Trisodium phosphate is readily soluble in water, with an alkaline reaction, and is prepared by evaporatively concentrating a solution of precisely 1 mol of disodium phosphate and 1 mol of NaOH. Tripotassium phosphate (tertiary or tribasic potassium phosphate), K₃PO₄, is a white, deliquescent, granular powder of density 2.56 gcm⁻³, has a melting point of 1340° and is readily soluble in water with an alkaline reaction. It is produced, for example, when Thomas slag is heated with charcoal and potassium sulfate. Despite the relatively high cost, the more readily soluble and therefore highly active potassium phosphates are frequently preferred in the cleaning products industry over corresponding sodium compounds.

Tetrasodium diphosphate(sodium pyrophosphate), Na₄P₂O₇, exists in anhydrous form (density 2.534 gcm⁻³, melting point 988°, 880° also reported) and in the form of the decahydrate (density 1.815-1.836 gcm⁻³, melting point 94° with loss of water) Both substances are colorless crystals which dissolve in water with an alkaline reaction. Na₄P₂O₇ is formed when disodium phosphate is heated to >200° or by reacting phosphoric acid with sodium carbonate in the stoichiometric ratio and dewatering the solution by spraying. The decahydrate complexes heavy metal salts and hardness formers and therefore reduces the hardness of water. Potassium diphosphate (potassium pyrophosphate), K₄P₂O₇, exists in the form of the trihydrate and is a colorless, hygroscopic powder of density 2.33 gcm⁻³, which is soluble in water, the pH of the 1% solution at 25° being 10.4.

Condensation of NaH₂PO₄ or of KH₂PO₄ gives rise to higher molecular weight sodium phosphates and potassium phosphates, for which a distinction can be drawn between cyclic representatives, the sodium metaphosphates and potassium metaphosphates, and catenated types, the sodium polyphosphates and potassium polyphosphates. For the latter in particular a multitude of names are in use: fused or calcined phosphates, Graham's salt, Kurrol's salt and Maddrell's salt. All higher sodium and potassium phosphates are referred to collectively as condensed phosphates.

The industrially important pentasodium triphosphate, Na₅P₃O₁₀ (sodium tripolyphosphate), is a nonhygroscopic, white, water-soluble salt which is anhydrous or crystallizes with 6 H₂O and has the general formula NaO—[P(O) (ONa)—O]ₙ—Na where n=3. About 17 g of the salt which is free of water of crystallization dissolve in 100 g of water at room temperature, at 60° approx. 20 g, at 100° around 32 g; after the solution has been heated at 100° for two hours, hydrolysis forms about 8% orthophosphate and 15% diphosphate. In the preparation of pentasodium triphosphate, phosphoric acid is reacted with sodium carbonate solution or sodium hydroxide solution in the stoichiometric ratio and the solution is dewatered by spraying. In a similar way to Graham's salt and sodium diphosphate, pentasodium triphosphate dissolves many insoluble metal compounds (including lime soaps etc.). Pentapotassium triphosphate, K₅P₃O₁₀ (potassium tripolyphosphate), is available commercially, for example, in the form of a 50% by weight solution (>23% P₂O₅, 25% K₂O). The potassium polyphosphates find wide use in the laundry detergents and cleaning products industry. There also exist sodium potassium tripolyphosphates which can likewise be used in the context of the present invention. They are formed, for example, when sodium trimetaphosphate is hydrolyzed with KOH:
(NaPO₃)₃+2 KOH→Na₃K₂P₃O₁₀+H₂O.

They can be used in accordance with the invention in precisely the same way as sodium tripolyphosphate, potassium tripolyphosphate or mixtures of the two; mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate can also be used in accordance with the invention.

Laundry detergent and cleaning composition tablets which are preferred in the context of the present invention are characterized in that the base tablet comprises phosphate(s), preferably alkali metal phosphate(s), more preferably pentasodium triphosphate or pentapotassium triphosphate (sodium tripolyphosphate or potassium tripolyphosphate), in amounts of from 20 to 80% by weight, preferably from 25 to 75% by weight and in particular from 30 to 70% by weight, based in each case on the weight of the base tablet.

Alkali carriers may be present as further constituents. Alkali carriers include alkali metal hydroxides, alkali metal carbonates, alkali metal hydrogencarbonates, alkali metal sesquicarbonates, alkali metal silicates, alkali metal metasilicates and mixtures of the aforementioned substances, and preference is given in the context of this invention to using the alkali metal carbonates, especially sodium carbonate, sodium hydrogencarbonate or sodium sesquicarbonate. Particular preference is given to a builder system comprising a mixture of tripolyphosphate and sodium carbonate. Particular preference is likewise given to a builder system comprising a mixture of tripolyphosphate and sodium carbonate and sodium disilicate.

In particularly preferred laundry detergent and cleaning composition tablets, the base tablet comprises carbonate(s) and/or hydrogencarbonate(s), preferably alkali metal carbonates, more preferably sodium carbonate, in amounts of from 5 to 50% by weight, preferably from 7.5 to 40% by weight and in particular from 10 to 30% by weight, based in each case on the weight of the base tablet.

Organic cobuilders which may find use in the inventive laundry detergent and cleaning composition tablets are in particular polycarboxylates/polycarboxylic acids, polymeric polycarboxylates, aspartic acid, polyacetals, dextrins, further organic cobuilders (see below) and phosphonates. These substance classes are described below.

Organic builder substances which can be used are, for example, the polycarboxylic acids usable in the form of their sodium salts, polycarboxylic acids referring to those carboxylic acids which bear more than one acid function. Examples of these are citric acid, adipic acid, succinic acid, glutaric acid, malic acid, tartaric acid, maleic acid, fumaric acid, sugar acids, aminocarboxylic acids, nitrilotriacetic acid (NTA), as long as such a use is not objectionable on ecological grounds, and mixtures thereof. Preferred salts are the salts of the polycarboxylic acids such as citric acid, adipic acid, succinic acid, glutaric acid, tartaric acid, sugar acids and mixtures thereof.

The acids themselves may also be used. In addition to their builder action, the acids typically also have the property of an acidifying component and thus also serve to set a lower and milder pH of laundry detergents and cleaning compositions. In this connection, particular mention should be made of citric acid, succinic acid, glutaric acid, adipic acid, gluconic acid and any mixtures thereof.

Also suitable as builders are polymeric polycarboxylates; these are, for example, the alkali metal salts of polyacrylic acid or of polymethacrylic acid, for example those having a relative molecular mass of from 500 to 70 000 g/mol.

In the context of this document, the molar masses specified for polymeric polycarboxylates are weight-average molar masses M_{w} of the particular acid form, which has always been determined by means of gel-permeation chromatography (GPC) using a UV detector. The measurement was against an external polyacrylic acid standard which, owing to its structural similarity to the polymers under investigation, provides realistic molecular weight values. These figures deviate considerably from the molecular weight data when polystyrenesulfonic acids are used as the standard. The molar masses measured against polystyrenesulfonic acids are generally distinctly higher than the molar masses specified in this document.

Suitable polymers are in particular polyacrylates which preferably have a molecular mass of from 2000 to 20 000 g/mol. Owing to their superior solubility, preference within this group may be given in turn to the short-chain polyacrylates which have molar masses of from 2000 to 10 000 g/mol and more preferably from 3000 to 5000 g/mol.

Also suitable are copolymeric polycarboxylates, especially those of acrylic acid with methacrylic acid and of acrylic acid or methacrylic acid with maleic acid. Copolymers which have been found to be particularly suitable are those of acrylic acid with maleic acid which contain from 50 to 90% by weight of acrylic acid and 50 to 10% by weight of maleic acid. Their relative molecular mass, based on free acids, is generally from 2000 to 70 000 g/mol, preferably from 20 000 to 50 000 g/mol and in particular from 30 000 to 40 000 g/mol.

The (co)polymeric polycarboxylates can either be used in the form of powders or in the form of aqueous solutions. The (co)polymeric polycarboxylate content of the compositions is preferably from 0.5 to 20% by weight, in particular from 3 to 10% by weight.

To improve the water solubility, the polymers may also contain allylsulfonic acids, for example allyloxybenzenesulfonic acid and methallylsulfonic acid, as monomers.

Also especially preferred are biodegradable polymers composed of more than two different monomer units, for example those which contain, as monomers, salts of acrylic acid or of maleic acid, and vinyl alcohol or vinyl alcohol derivatives, or those which contain, as monomers, salts of acrylic acid and of 2-alkylallylsulfonic acid, and sugar derivatives.

Further preferred copolymers are those which preferably have, as monomers, acrolein and acrylic acid/acrylic acid salts or acrolein and vinyl acetate.

Further preferred builder substances which should likewise be mentioned are polymeric aminodicarboxylic acids, salts thereof or precursor substances thereof. Particular preference is given to polyaspartic acids or salts and derivatives thereof.

Further suitable builder substances are polyacetals which can be obtained by reacting dialdehydes with polyolcarboxylic acids which have from 5 to 7 carbon atoms and at least 3 hydroxyl groups. Preferred polyacetals are obtained from dialdehydes such as glyoxal, glutaraldehyde, terephthalaldehyde, and mixtures thereof, and from polyolcarboxylic acids such as gluconic acid and/or glucoheptonic acid.

Further suitable organic builder substances are dextrins, for example oligomers or polymers of carbohydrates, which can be obtained by partial hydrolysis of starches. The hydrolysis can be carried out by customary, for example acid-catalyzed or enzyme-catalyzed, processes. The hydrolysis products preferably have average molar masses in the range from 400 to 500 000 g/mol. Preference is given to a polysaccharide having a dextrose equivalent (DE) in the range from 0.5 to 40, in particular from 2 to 30, where DE is a common measure of the reducing action of a polysaccharide compared to dextrose, which has a DE of 100. It is also possible to use maltodextrins with a DE between 3 and 20 and dry glucose syrups with a DE between 20 and 37, and also yellow dextrins and white dextrins having relatively high molar masses in the range from 2000 to 30 000 g/mol.

The oxidized derivatives of such dextrins are their reaction products with oxidizing agents which are capable of oxidizing at least one alcohol function of the saccharide ring to the carboxylic acid function.

Oxydisuccinates and other derivatives of disuccinates, preferably ethylenediaminedisuccinate, are also further suitable cobuilders. In this case, ethylenediamine N,N′-disuccinate (EDDS) is preferably used in the form of its sodium or magnesium salts. In this connection, preference is also given to glyceryl disuccinates and glyceryl trisuccinates. Suitable use amounts in zeolite-containing and/or silicate-containing formulations are from 3 to 15% by weight.

Further organic cobuilders which can be used are, for example, acetylated hydroxycarboxylic acids or salts thereof, which may also be present in lactone form and which contain at least 4 carbon atoms and at least one hydroxyl group and a maximum of two acid groups.

A further class of substances having cobuilder properties is that of the phosphonates. These are in particular hydroxyalkane- and aminoalkanephosphonates. Among the hydroxyalkanephosphonates, 1-hydroxyethane-1,1-diphosphonate (HEDP) is of particular significance as a cobuilder. It is preferably used in the form of the sodium salt, the disodium salt giving a neutral reaction and the tetrasodium salt an alkaline reaction (pH 9). Useful aminoalkanephosphonates are preferably ethylenediaminetetramethylenephosphonate (EDTMP), diethylenetriaminepentamethylenephosphonate (DTPMP) and higher homologs thereof. They are preferably used in the form of the neutrally reacting sodium salts, for example as the hexasodium salt of EDTMP or as the hepta- and octasodium salt of DTPMP. From the class of the phosphonates, preference is given to using HEDP as a builder. In addition, the aminoalkanephosphonates have a marked heavy metal-binding capacity. Accordingly, especially when the compositions also comprise bleaches, it may be preferable to use aminoalkanephosphonates, especially DTPMP, or mixtures of the phosphonates mentioned.

In addition, it is also possible to use all compounds which are capable of forming complexes with alkaline earth metal ions as cobuilders.

The amount of builders is typically between 10 and 70% by weight, preferably between 15 and 60% by weight and in particular between 20 and 50% by weight, based in each case on the base tablet.

In addition to builder(s), the inventive compositions may preferably further comprise, in the base tablet, as a surfactant component, anionic, nonionic, cationic and/or amphoteric surfactants, preference being given to nonionic surfactants owing to their foaming capacity.

The anionic surfactants used are, for example, those of the sulfonate and sulfate type. Useful surfactants of the sulfonate type are preferably C₉₋₁₃-alkylbenzenesulfonates, olefinsulfonates, i.e. mixtures of alkene- and hydroxyalkanesulfonates, and disulfonates, as are obtained, for example, from C₁₂₋₁₈-monoolefins with terminal or internal double bond by sulfonation with gaseous sulfur trioxide and subsequent alkaline or acidic hydrolysis of the sulfonation products. Also suitable are alkanesulfonates which are obtained from C₁₂₋₁₈-alkanes, for example by sulfochlorination or sulfoxidation with subsequent hydrolysis or neutralization. The esters of α-sulfo fatty acids (ester sulfonates), for example the α-sulfonated methyl esters of hydrogenated coconut, palm kernel or tallow fatty acids, are also likewise suitable.

Further suitable anionic surfactants are sulfated fatty acid glycerol esters. Fatty acid glycerol esters refer to the mono-, di- and triesters, and mixtures thereof, as are obtained in the preparation by esterification of a monoglycerol with from 1 to 3 mol of fatty acid or in the transesterification of triglycerides with from 0.3 to 2 mol of glycerol. Preferred sulfated fatty acid glycerol esters are the sulfation products of saturated fatty acids having from 6 to 22 carbon atoms, for example of caproic acid, caprylic acid, capric acid, myristic acid, lauric acid, palmitic acid, stearic acid or behenic acid.

Preferred alk(en)yl sulfates are the alkali metal and in particular the sodium salts of the sulfuric monoesters of C₁₂-C₁₈ fatty alcohols, for example of coconut fatty alcohol, tallow fatty alcohol, lauryl, myristyl, cetyl or stearyl alcohol, or of C₁₀-C₂₀ oxo alcohols and those monoesters of secondary alcohols of these chain lengths. Also preferred are alk(en)yl sulfates of the chain length mentioned which contain a synthetic straight-chain alkyl radical prepared on a petrochemical basis and which have analogous degradation behavior to the equivalent compounds based on fatty chemical raw materials. From the washing point of view, preference is given to the C₁₂-C₁₆-alkyl sulfates and C₁₂-C₁₅-alkyl sulfates, and C₁₄-C₁₅-alkyl sulfates. 2,3-Alkyl sulfates, which can be obtained as commercial products from the Shell Oil Company under the name DAN®, are also suitable anionic surfactants.

Also suitable are the sulfuric monoesters of the straight-chain or branched C₇₋₂₁-alcohols ethoxylated with 1 to 6 mol of ethylene oxide, such as 2-methyl-branched C₉₋₁₁-alcohols with on average 3.5 mol of ethylene oxide (EO) or C₁₂₋₁₈-fatty alcohols with from 1 to 4 EO. Owing to their high tendency to foam, they are used in detergents only in relatively small amounts, for example amounts of from 1 to 5% by weight.

Further suitable anionic surfactants are also the salts of alkylsulfosuccinic acid, which are also referred to as sulfosuccinates or as sulfosuccinic esters and are the monoesters and/or diesters of sulfosuccinic acid with alcohols, preferably fatty alcohols and in particular ethoxylated fatty alcohols. Preferred sulfosuccinates contain C₈₋₁₈ fatty alcohol radicals or mixtures thereof. Especially preferred sulfosuccinates contain a fatty alcohol radical which is derived from ethoxylated fatty alcohols which, considered alone, constitute nonionic surfactants (for description see below). In this context, particular preference is again given to sulfosuccinates whose fatty alcohol radicals are derived from ethoxylated fatty alcohols with a narrowed homolog distribution. It is also equally possible to use alk(en)ylsuccinic acid having preferably from 8 to 18 carbon atoms in the alk(en)yl chain or salts thereof.

Suitable further anionic surfactants are in particular soaps. Suitable soaps are saturated fatty acid soaps, such as the salts of lauric acid, myristic acid, palmitic acid, stearic acid, hydrogenated erucic acid and behenic acid, and soap mixtures derived in particular from natural fatty acids, for example coconut, palm kernel or tallow fatty acids.

The anionic surfactants including the soaps may be present in the form of their sodium, potassium or ammonium salts, and also in the form of soluble salts of organic bases, such as mono-, di- or triethanolamine. The anionic surfactants are preferably present in the form of their sodium or potassium salts, in particular in the form of the sodium salts.

The nonionic surfactants used are preferably alkoxylated, advantageously ethoxylated, in particular primary alcohols having preferably from 8 to 18 carbon atoms and on average from 1 to 12 mol of ethylene oxide (EO) per mole of alcohol in which the alcohol radical may be linear or preferably 2-methyl-branched, or may contain a mixture of linear and methyl-branched radicals, as are typically present in oxo alcohol radicals. However, especially preferred alcohol ethoxylates have linear radicals of alcohols of natural origin having from 12 to 18 carbon atoms, for example of coconut, palm, tallow fat or oleyl alcohol, and on average from 2 to 8 EO per mole of alcohol. The preferred ethoxylated alcohols include, for example, C₁₂₋₁₄-alcohols having 3 EO or 4 EO, C₉₋₁₁-alcohol having 7 EO, C₁₃₋₁₅-alcohols having 3 EO, 5 EO, 7 EO or 8 EO, C₁₂₋₁₈-alcohols having 3 EO, 5 EO or 7 EO and mixtures thereof, such as mixtures of C₁₂₋₁₄-alcohol having 3 EO and C₁₂₋₁₈-alcohol having 5 EO. The degrees of ethoxylation specified are statistical average values which may be an integer or a fraction for a specific product. Preferred alcohol ethoxylates have a narrowed homolog distribution (narrow range ethoxylates, NRE). In addition to these nonionic surfactants, it is also possible to use fatty alcohols having more than 12 EO. Examples thereof are tallow fatty alcohol having 14 EO, 25 EO, 30 EO or 40 EO.

In addition, further nonionic surfactants which may be used are also alkyl glycosides of the general formula RO(G)ₓ in which R is a primary straight-chain or methyl-branched, in particular 2-methyl-branched, aliphatic radical having from 8 to 22, preferably from 12 to 18, carbon atoms and G is the symbol which is a glycose unit having 5 or 6 carbon atoms, preferably glucose. The degree of oligomerization x, which specifies the distribution of monoglycosides and oligoglycosides, is any number between 1 and 10; x is preferably from 1.2 to 1.4.

A further class of nonionic surfactants used with preference, which are used either as the sole nonionic surfactant or in combination with other nonionic surfactants, are alkoxylated, preferably ethoxylated or ethoxylated and propoxylated, fatty acid alkyl esters, preferably having from 1 to 4 carbon atoms in the alkyl chain.

Nonionic surfactants of the amine oxide type, for example N-cocoalkyl-N,N-dimethylamine oxide and N-tallow alkyl-N,N-dihydroxyethylamine oxide, and of the fatty acid alkanolamide type may also be suitable. The amount of these nonionic surfactants is preferably not more than that of the ethoxylated fatty alcohols, in particular not more than half thereof.

Further suitable surfactants are polyhydroxy fatty acid amides of the formula (I) 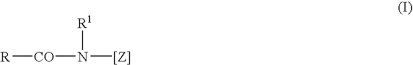
in which RCO is an aliphatic acyl radical having from 6 to 22 carbon atoms, R¹ is hydrogen, an alkyl or hydroxyalkyl radical having from 1 to 4 carbon atoms and [Z] is a linear or branched polyhydroxyalkyl radical having from 3 to 10 carbon atoms and from 3 to 10 hydroxyl groups. The polyhydroxy fatty acid amides are known substances which can typically be obtained by reductively aminating a reducing sugar with ammonia, an alkylamine or an alkanolamine, and subsequently acylating with a fatty acid, a fatty acid alkyl ester or a fatty acid chloride.

The group of polyhydroxy fatty acid amides also includes compounds of the formula (II) 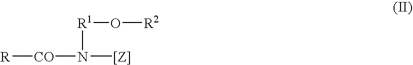
in which R is a linear or branched alkyl or alkenyl radical having from 7 to 12 carbon atoms, R¹ is a linear, branched or cyclic alkyl radical or an aryl radical having from 2 to 8 carbon atoms and R² is a linear, branched or cyclic alkyl radical or an aryl radical or an oxyalkyl radical having from 1 to 8 carbon atoms, preference being given to C₁₋₄-alkyl or phenyl radicals, and [Z] is a linear polyhydroxyalkyl radical whose alkyl chain is substituted by at least two hydroxyl groups, or alkoxylated, preferably ethoxylated or propoxylated, derivatives of this radical.

[Z] is preferably obtained by reductive amination of a reduced sugar, for example glucose, fructose, maltose, lactose, galactose, mannose or xylose. The N-alkoxy- or N-aryloxy-substituted compounds can be converted to the desired polyhydroxy fatty acid amides by reaction with fatty acid methyl esters in the presence of an alkoxide as catalyst.

The surfactants used with preference are low-foaming nonionic surfactants. With particular preference, the inventive cleaning compositions for machine dishwashing comprise nonionic surfactants, in particular nonionic surfactants from the group of the alkoxylated alcohols. The nonionic surfactants used are preferably alkoxylated, advantageously ethoxylated, in particular primary alcohols having preferably from 8 to 18 carbon atoms and on average from 1 to 12 mol of ethylene oxide (EO) per mole of alcohol in which the alcohol radical may be linear or preferably 2-methyl-branched, or may contain a mixture of linear and methyl-branched radicals, as are typically present in oxo alcohol radicals. However, especially preferred alcohol ethoxylates have linear radicals of alcohols of natural origin having from 12 to 18 carbon atoms, for example of coconut, palm, tallow fat or oleyl alcohol, and on average from. 2 to 8 EO per mole of alcohol. The preferred ethoxylated alcohols include, for example, C₁₂₋₁₄-alcohols having 3 EO or 4 EO, C₉₋₁₁-alcohol having 7 EO, C₁₃₋₁₅-alcohols having 3 EO, 5 EO, 7 EO or 8 EO, C₁₂₋₁₈-alcohols having 3 EO, 5 EO or 7 EO and mixtures thereof, such as mixtures of C₁₂₋₁₄-alcohol having 3 EO and C₁₂₋₁₈-alcohol having 5 EO. The degrees of ethoxylation specified are statistical average values which may be an integer or a fraction for a specific product. Preferred alcohol ethoxylates have a narrowed homolog distribution (narrow range ethoxylates, NRE). In addition to these nonionic surfactants, it is also possible to use fatty alcohols having more than 12 EO. Examples thereof are tallow fatty alcohol having 14 EO, 25 EO, 30 EO or 40 EO.

Special preference is given to inventive dishwasher detergents which comprise a nonionic surfactant which has a melting point above room temperature. Accordingly, preferred dishwasher detergents are characterized in that they comprise nonionic surfactant(s) having a melting point above 20° C., preferably above 25° C., more preferably between 25 and 60° C. and in particular between 26.6 and 43.3° C.

Suitable nonionic surfactants which have melting or softening points in the temperature range specified are, for example, low-foaming nonionic surfactants which may be solid or highly viscous at room temperature. When nonionic surfactants which have a high viscosity at room temperature are used, they preferably have a viscosity above 20 Pas, preferably above 35 Pas and in particular above 40 Pas. Nonionic surfactants which have a waxlike consistency at room temperature are also preferred.

Nonionic surfactants which are solid at room temperature and are to be used with preference stem from the group of alkoxylated nonionic surfactants, in particular the ethoxylated primary alcohols and mixtures of these surfactants with structurally complex surfactants, such as polyoxypropylene/polyoxyethylene/polyoxypropylene (PO/EO/PO) surfactants. Such (PO/EO/PO) nonionic surfactants are additionally notable for good foam control.

In a preferred embodiment of the present invention, the nonionic surfactant with a melting point above room temperature is an ethoxylated nonionic surfactant which has resulted from the reaction of a monohydroxyalkanol or alkylphenol having from 6 to 20 carbon atoms with preferably at least 12 mol, more preferably at least 15 mol, in particular at least 20 mol, of ethylene oxide per mole of alcohol or alkylphenol.

A nonionic surfactant which is solid at room temperature and is to be used with particular preference is obtained from a straight-chain fatty alcohol having from 16 to 20 carbon atoms (C₁₆₋₂₀-alcohol), preferably a C₁₈-alcohol, and at least 12 mol, preferably at least 15 mol and in particular at least 20 mol, of ethylene oxide. Of these, the “narrow range ethoxylates” (see above) are particularly preferred.

Accordingly, particularly preferred inventive dishwasher detergents comprise ethoxylated nonionic surfactant(s) which has/have been obtained from C₆₋₂₀-monohydroxyalkanols or C₆₋₂₀-alkylphenols or C₁₆₋₂₀-fatty alcohols and more than 12 mol, preferably more than 15 mol and in particular more than 20 mol, of ethylene oxide per mole of alcohol.

The room temperature solid nonionic surfactant preferably additionally has propylene oxide units in the molecule. Preferably, such PO units make up up to 25% by weight, more preferably up to 20% by weight and in particular up to 15% by weight, of the total molar mass. of the nonionic surfactant. Particularly preferred nonionic surfactants are ethoxylated monohydroxyalkanols or alkylphenols which additionally have polyoxyethylene-polyoxypropylene block copolymer units. The alcohol or alkylphenol moiety of such nonionic surfactant molecules preferably makes up more than 30% by weight, more preferably more than 50% by weight and in particular more than 70% by weight, of the total molar mass of such nonionic surfactants. Preferred dishwasher detergents are characterized in that they comprise ethoxylated and propoxylated nonionic surfactants in which the propylene oxide units in the molecule make up up to 25% by weight, preferably up to 20% by weight and in particular up to 15% by weight, of the total molar mass of the nonionic surfactant.

Further nonionic surfactants which have melting points above room temperature and are to be used with particular preference contain from 40 to 70% of a polyoxypropylene/polyoxyethylene/polyoxypropylene block polymer blend which contains 75% by weight of an inverse block copolymer of polyoxyethylene and polyoxypropylene having 17 mol of ethylene oxide and 44 mol of propylene oxide, and 25% by weight of a block copolymer of polyoxyethylene and polyoxypropylene initiated with trimethylolpropane and containing 24 mol of ethylene oxide and 99 mol of propylene oxide per mole of trimethylolpropane.

Nonionic surfactants which can be used with particular preference are obtainable, for example, under the name Poly Tergent® SLF-18 from Olin Chemicals.

A further preferred inventive dishwasher detergent comprises nonionic surfactants of the formula
R¹O[CH₂CH(CH₃)O]ₓCH₂CH₂O]_{y}[CH₂CH(OH)R²]
in which R¹ is a linear or branched aliphatic hydrocarbon radical having from 4 to 18 carbon atoms or mixtures thereof, R² is a linear or branched hydrocarbon radical having from 2 to 26 carbon atoms or mixtures thereof, and x is a value between 0.5 and 1.5, and y is a value of at least 15.

Further nonionic surfactants which can be used with preference are the terminally capped poly(oxyalkylated) nonionic surfactants of the formula
R¹O[CH₂CH(R³)O]ₓ[CH₂]ₖCH(OH)[CH₂]ⱼOR²
in which R¹ and R² are linear or branched, saturated or unsaturated, aliphatic or aromatic hydrocarbon radicals having from 1 to 30 carbon atoms, R³ is H or a methyl, ethyl, n-propyl, isopropyl, n-butyl, 2-butyl or 2-methyl-2-butyl radical, x is a value between 1 and 30, k and j are values between 1 and 12, preferably between 1 and 5. When the value x is ≧2, each R³ in the above formula may be different. R¹ and R² are preferably linear or branched, saturated or unsaturated, aliphatic or aromatic hydrocarbon radicals having from 6 to 22 carbon atoms, particular preference being given to radicals having from 8 to 18 carbon atoms. For the R³ radical, particular preference is given to H, —CH₃ or —CH₂CH₃. Particularly preferred values for x are in the range from 1 to 20, in particular from 6 to 15.

As described above, each R³ in the above formula may be different if x is ≧2. This allows the alkylene oxide unit in the square brackets to be varied. When x is, for example, 3, the R³ radical may be selected so as to form ethylene oxide (R³═H) or propylene oxide (R³═CH₃) units which can be joined together in any sequence, for example (EO) (PO) (EO), (EO) (EO) (PO), (EO) (EO) (EO), (PO) (EO) (PO), (PO) (PO) (EO) and (PO) (PO) (PO). The value 3 for x is selected here by way of example and it is entirely possible for it to be larger, the scope of variation increasing with increasing x values and embracing, for example, a large number of (EO) groups combined with a small number of (PO) groups, or vice versa.

Especially preferred terminally capped poly(oxyalkylated) alcohols of the above formula have values of k=1 and j=1, so that the above formula is simplified to
R¹O[CH₂CH(R³)O]ₓCH₂CH(OH)CH₂OR².

In the latter formula, R¹, R² and R³ are each as defined above and x is a number from 1 to 30, preferably from 1 to 20 and in particular from 6 to 18. Particular preference is given to surfactants in which the R¹ and R² radicals have from 9 to 14 carbon atoms, R³ is H and x assumes values of from 6 to 15.

If the latter statements are summarized, preference is given to inventive dishwasher detergents which comprise terminally capped poly(oxyalkylated) nonionic surfactants of the formula
R¹O[CH₂CH(R³)O]ₓ[CH₂]ₖCH(OH) [CH₂]ⱼOR²
in which R¹ and R² are linear or branched, saturated or unsaturated, aliphatic or aromatic hydrocarbon radicals having from 1 to 30 carbon atoms, R³ is H or a methyl, ethyl, n-propyl, isopropyl, n-butyl, 2-butyl or 2-methyl-2-butyl radical, x is a value between 1 and 30, k and j are values between 1 and 12, preferably between 1 and 5, particular preference being given to surfactants of the
R¹O[CH₂CH(R³)O]ₓCH₂CH(OH)CH₂OR²
type in which x is a number from 1 to 30, preferably from 1 to 20 and in particular from 6 to 18.

Particularly preferred nonionic surfactants in the context of the present invention have been found to be low-foaming nonionic surfactants which have alternating ethylene oxide and alkylene oxide units. Among these, preference is given in turn to surfactants having EO-AO-EO-AO blocks, and in each case from 1 to 10 EO and/or AO groups are bonded to one another before a block of the other groups in each case follows. Preference is given here to inventive machine dishwasher detergents which comprise, as nonionic surfactant(s), surfactants of the general formula III 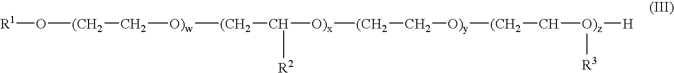
in which R¹ is a straight-chain or branched, saturated or mono- or polyunsaturated C₆₋₂₄-alkyl or -alkenyl radical; each R² or R³ group is independently selected from —CH₃; —CH₂CH₃, —CH₂CH₂—CH₃, —CH(CH₃)₂ and the indices w, x, y, z are each independently integers from 1 to 6.

The preferred nonionic surfactants of the formula III can be prepared by known methods from the corresponding alcohols R¹—OH and ethylene oxide or alkylene oxide. The R¹ radical in the above formula X may vary depending on the origin of the alcohol. When native sources are utilized, the R¹ radical has an even number of carbon atoms and is generally unbranched, and preference is given to the linear radicals of alcohols of native origin having from 12 to 18 carbon atoms, for example from coconut, palm, tallow fat or oleyl alcohol. Alcohols obtainable from synthetic sources are, for example, the Guerbet alcohols or 2-methyl-branched or linear and methyl-branched radicals in a mixture, as are typically present in oxo alcohol radicals. Irrespective of the type of the alcohol used to prepare the nonionic surfactants present in accordance with the invention in the products, preference is given to inventive machine dishwasher detergents in which R¹ in formula I is an alkyl radical having from 6 to 24, preferably from 8 to 20, more preferably 9 to 15 and in particular 9 to 11 carbon atoms.

The alkylene oxide unit which is present in the preferred nonionic surfactants in alternation to the ethylene oxide unit is, as well as propylene oxide, especially butylene oxide. However, further alkylene oxides in which R² and R³ are each independently selected from —CH₂CH₂—CH₃ and —CH(CH₃)₂ are also suitable. Preferred machine dishwasher detergents are characterized in that R² and R³ are each a —CH₃ radical, w and x are each independently 3 or 4, and y and z are each independently 1 or 2.

In summary, preference is given for use in the inventive compositions especially to nonionic surfactants which have a C₉₋₁₅ alkyl radical having from 1 to 4 ethylene oxide units, followed by from 1 to 4 propylene oxide units, followed by from 1 to 4 ethylene oxide units, followed by from 1 to 4 propylene oxide units. In aqueous solution, these surfactants have the required low viscosity and can be used with particular preference in accordance with the invention.

The specified carbon chain lengths and degrees of ethoxylation or degrees of alkoxylation constitute statistical averages which may be a whole number or a fraction for a specific product. As a consequence of the preparation process, commercial products of the formulae specified do not usually consist of one individual representative, but rather of mixtures, as a result of which average values and consequently fractions can arise both for the carbon chain lengths and for the degrees of ethoxylation or degrees of alkoxylation.

Instead of the surfactants mentioned or in conjunction with them, it is also possible to use cationic and/or amphoteric surfactants.

As cationic active substances, the inventive compositions may, for example, comprise cationic compounds of the formulae IV, V or VI: 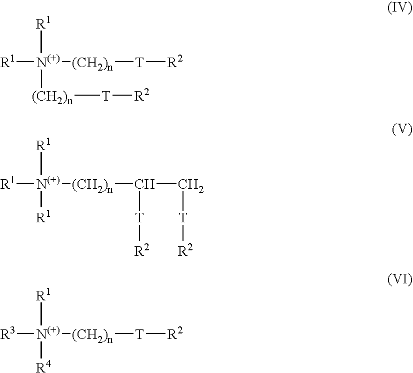
in which each R¹ group is independently selected from C₁₋₆-alkyl, -alkenyl or -hydroxyalkyl groups; each R² group is independently selected from C₈₋₂₈-alkyl or -alkenyl groups; R³═R¹ or (CH₂)ₙ-T-R²; R⁴═R¹ or R² or (CH₂)ₙ-T-R²; T=-CH₂—, —O—CO— or —CO—O— and n is an integer from 0 to 5.

In summary, preference is given to inventive depression tablets which contain surfactant(s), preferably nonionic surfactant(s), and in particular nonionic surfactant(s) from the group of the alkoxylated alcohols, in amounts of from 0.1 to 60% by weight, preferably from 0.5 to 50% by weight, more preferably from 1 to 40% by weight and in particular from 2 to 30% by weight, based in each case on the overall composition. Particularly preferred inventive machine laundry detergent or cleaning composition tablets are characterized in that they contain from 5 to 25% by weight, preferably from 6 to 22.5% by weight, more preferably from 7.5 to 20% by weight and in particular from 8 to 17.5% by weight, of nonionic surfactant(s)

In addition to the builders, preferred ingredients of inventive laundry detergent or cleaning composition tablets are in particular bleaches, bleach activators, enzymes, silver protectants, dyes and fragrances, etc. In addition, further ingredients may be present, preference being given to inventive laundry detergent or cleaning composition tablets which additionally comprise one or more substances from the group of the acidifiers or the chelate complexing agents.

Useful acidifiers are both inorganic acids and also organic acids, as long as they are compatible with the other ingredients. For reasons of consumer protection and of handling safety, the solid mono-, oligo- and polycarboxylic acids in particular can be used. From this group, preference is given in turn to citric acid, tartaric acid, succinic acid, malonic acid, adipic acid, maleic acid, fumaric acid, oxalic acid, and polyacrylic acid. The anhydrides of these acids can also be used as acidifiers, and maleic anhydride and succinic anhydride in particular are commercially available. Organic sulfonic acids, such as amidosulfonic acid, can likewise be used. A substance which is commercially available and can likewise be used with preference as an acidifier in the context of the present invention is Sokalan® DCS (trademark of BASF), a mixture of succinic acid (max. 31% by weight), glutaric acid (max. 50% by weight) and adipic acid (max. 33% by weight).

A further possible group of ingredients is that of the chelate complexing agents. Chelate complexing agents are substances which form cyclic compounds with metal ions, an individual ligand occupying more than one coordination site on a central atom, i.e. being at least “bidentate”. In this case, normally extended compounds are thus closed to give rings by complex formation via an ion. The number of bound ligands depends on the coordination number of the central ion.

Chelate complexing agents which are commonly used and preferred in the context of the present invention are, for example, polyoxycarboxylic acids, polyamines, ethylenediaminetetraacetic acid (EDTA) and nitrilotriacetic acid (NTA). Also usable in accordance with the invention are complex-forming polymers, i.e. polymers which bear functional groups either in the main chain itself or pendent to it, which can act as ligands and react with suitable metal atoms generally to form chelate complexes. The polymer-bound ligands of the resulting metal complexes can stem from just one macromolecule or else belong to different polymer chains. The latter leads to the crosslinking of the material when the complex-forming polymers have not already been crosslinked beforehand via covalent bonds.

Complexing groups (ligands) of customary complex-forming polymers are iminodiacetic acid, hydroxyquinoline, thiourea, guanidine, dithiocarbamate, hydroxamic acid, amidoxime, aminophosphoric acid, (cyclic) polyamino, mercapto, 1,3-dicarbonyl and crown ether radicals, some of which have very specific activities toward ions of different metals. Basis polymers of many complex-forming polymers, which are also commercially significant, are polystyrene, polyacrylates, polyacrylonitriles, polyvinyl alcohols, polyvinylpyridines and polyethylenimines. Natural polymers, such as cellulose, starch or chitin are also complex-forming polymers. In addition, they may be provided with further ligand functionalities as a result of polymer-analogous modifications.

In the context of the present invention, particular preference is given to laundry detergent or cleaning composition tablets, in particular to machine dishwasher detergent tablets, which contain one or more chelate complexing agents from the groups of
(i) polycarboxylic acids in which the sum of the carboxyl and any hydroxyl groups is at least 5,
(ii) nitrogen-containing mono- or polycarboxylic acids,
(iii) geminal diphosphonic acids,
(iv) aminophosphonic acids,
(v) phosphonopolycarboxylic acids,
(vi) cyclodextrins
   in amounts above 0.1% by weight, preferably above 0.5% by weight, more preferably above 1% by weight and in particular above 2.5% by weight, based in each case on the weight of the dishwasher detergent.

In the context of the present invention, it is possible to use all complexing agents of the prior art. These may belong to different chemical groups. Preference is given to using the following, individually or in a mixture with one another:
a) polycarboxylic acids in which the sum of the carboxyl and any hydroxyl groups is at least 5, such as gluconic acid,
b) nitrogen-containing mono- or polycarboxylic acids, such as ethylenediaminetetraacetic acid (EDTA), N-hydroxyethylethylenediaminetriacetic acid, diethylenetriaminepentaacetic acid, hydroxyethyliminodiacetic acid, nitridodiacetic acid-3-propionic acid, isoserinediacetic acid, N,N-di(β-hydroxyethyl)glycine, N-(1,2-dicarboxy-2-hydroxyethyl)glycine, N-(1,2-dicarboxy-2-hydroxyethyl)-aspartic acid or nitrilotriacetic acid (NTA), c) geminal diphosphonic acids, such as 1-hydroxyethane-1,1-diphosphonic acid (HEDP), higher homologs thereof having up to 8 carbon atoms, and hydroxyl- or amino-containing derivatives thereof and 1-aminoethane-1,1-diphosphonic acid, higher homologs thereof having up to 8 carbon atoms, and hydroxyl- or amino-containing derivatives thereof,
d) aminophosphonic acids, such as ethylenediaminetetra(methylenephosphonic acid), diethylenetriaminepenta(methylenephosphonic acid) or nitrilotri-(methylenephosphonic acid),
e) phosphonopolycarboxylic acids, such as 2-phosphonobutane-1,2,4-tricarboxylic acid, and
f) cyclodextrins.

In the context of this patent application, polycarboxylic acids a) refer to carboxylic acids, including monocarboxylic acids, in which the sum of carboxyl and the hydroxyl groups present in the molecule is at least 5. Preference is given to complexing agents from the group of nitrogen-containing polycarboxylic acids, in particular EDTA. At the alkaline pH values of the treatment solutions required in accordance with the invention, these complexing agents are at least partially in the form of anions. It is unimportant whether they are introduced in the form of acids or in the form of salts. In the case of use in the form of salts, preference is given to alkali metal, ammonium or alkylammonium salts, in particular sodium salts.

The machine cleaning of tableware in domestic machine dishwashers typically includes a prewash cycle, a main wash cycle and rinse cycle, which are interrupted by intermediate wash cycles. In most machines, the prewash cycle can be selected for highly soiled tableware, but is only selected by the consumer in exceptional cases, so that a main wash cycle, an intermediate wash cycle with clean water and a rinse cycle are carried out in most machines. The temperature of the main wash cycle varies, depending on the machine type and program level selection, between 40 and 65° C. In the rinse cycle, rinse aids are added from a metering tank in the machine and typically comprise nonionic surfactants as the main constituent. Such rinse aids are present in liquid form and are widely described in the prior art. Their task consists primarily in preventing lime spots and films on the cleaned tableware. In addition to water and low-foaming nonionic surfactants, these rinse aids often also comprise hydrotropes, pH modifiers such as citric acid or film-inhibiting polymers.

The reservoir tank in the machine dishwasher has to be filled up with rinse aid at regular intervals, and one filling is sufficient for from 10 to 50 wash cycles depending on the machine type. When the user forgets to fill up the tank, glasses in particular acquire unsightly lime spots and films. In the prior art, there therefore exist some proposed solutions for the integration of a rinse aid into the detergent for machine dishwashing. “2-in-1” products which combine detergent and rinse aid in one have also become established on the market.

A further development has the aim of making it unnecessary even to fill up the regeneration salt vessel of the machine dishwasher. What are known as “3-in-1” detergents which combine the three functions of cleaning, of aiding the rinse and of softening the water in a single detergent formulation, so that the replenishment of salt in the case of water hardnesses of from 1 to 3 becomes superfluous to the consumer, have also come onto the market. For water softening, these detergents typically comprise sodium tripolyphosphate and/or polymers effective as softeners.

Inventive cleaning composition tablets for machine dishwashing may therefore additionally, preferably in the base tablet, comprise polymers effective as softeners. With particular preference, polymers containing sulfonic acid groups are used, and are described below.

Polymers which contain sulfonic acid groups and can be used with particular preference are copolymers of unsaturated carboxylic acids, monomers containing sulfonic acid groups and optionally further ionic or nonionogenic monomers.

In the context of the present invention, preference is given to unsaturated carboxylic acids of the formula VII as a monomer
R¹(R²)C═C(R³)COOH (VII)
in which R¹ to R³ are each independently —H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms.

Among the unsaturated carboxylic acids which can be described by the formula VII, preference is given in particular to acrylic acid (R¹═R²═R³═H), methacrylic acid (R¹═R²═H; R³═CH₃) and/or maleic acid (R¹═COOH; R²═R³═H).

The monomers containing sulfonic acid groups are preferably those of the formula VIII
R⁵ (R⁶)C═C(R⁷)—X—SO₃H (VIII)
in which R⁵ to R⁷ are each independently —H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms, and X is an optionally present spacer group which is selected from —(CH₂)ₙ— where n=from 0 to 4, —COO—(CH₂)ₖ— where k=from 1 to 6, —C(O)—NH—C(CH₃)₂— and —C(O)—NH—CH(CH₂CH₃)—.

Among these monomers, preference is given to those of the formulae VIIIa, VIIIb and/or VIIIc
H₂C═CH—X—SO₃H (VIIIa)
H₂C═C(CH₃)—X—SO₃H (VIIIb)
HO₃S—X—(R⁶)C═C(R⁷)—X—SO₃H (VIIIc)
in which R⁶ and R⁷ are each independently selected from —H, —CH₃, —CH₂CH₃, —CH₂CH₂CH₃, —CH(CH₃)₂ and X is an optionally present spacer group which is selected from —(CH₂)ₙ— where n=from 0 to 4, —COO—(CH₂)ₖ— where k=from 1 to 6, —C(O)—NH—C(CH₃)₂— and —C(O)—NH—CH(CH₂CH₃)—.

Particularly preferred monomers containing sulfonic acid groups are 1-acrylamido-1-propanesulfonic acid (X═—C(O)NH—CH(CH₂CH₃) in formula VIIIa), 2-acrylamido-2-propanesulfonic acid (X═—C(O)NH—C(CH₃)₂ in formula VIIIa), 2-acrylamido-2-methyl-1-propanesulfonic acid (X═—C(O)NH—CH(CH₃)CH₂— in formula VIIIa), 2-methacrylamido-2-methyl-1-propanesulfonic acid (X═—C(O)NH—CH(CH₃)CH₂— in formula VIIIb), 3-methacrylamido-2-hydroxypropanesulfonic acid (X═—C(O)NH—CH₂CH(OH)CH₂— in formula VIIIb), allylsulfonic acid (X═CH₂ in formula VIIIa), methallylsulfonic acid (X═CH₂ in formula VIIIb), allyloxybenzenesulfonic acid (X═—CH₂—O—C₆H₄— in formula VIIIa), methallyloxybenzenesulfonic acid (X═—CH₂—O—C₆H₄— in formula VIIIb), 2-hydroxy-3-(2-propenyloxy)propanesulfonic acid, 2-methyl-2-propene-1-sulfonic acid (X═CH₂ in formula VIIIb), styrenesulfonic acid (X═C₆H₄ in formula VIIIa), vinylsulfonic acid (X not present in formula VIIIa), 3-sulfopropyl acrylate (X═—C(O)NH—CH₂CH₂CH₂— in formula VIIIa), 3-sulfopropyl methacrylate (X═—C(O)NH—CH₂CH₂CH₂— in formula VIIIb), sulfomethacrylamide (X═—C(O)NH— in formula VIIIb), sulfomethylmethacrylamide (X═—C(O)NH—CH₂— in formula VIIIb) and water-soluble salts of the acids mentioned.

Useful further ionic or nonionogenic monomers are in particular ethylenically unsaturated compounds. The content of monomers of group iii) in the polymers used in accordance with the invention is preferably less than 20% by weight, based on the polymer. Polymers to be used with particular preference consist only of monomers of groups i) and ii).

In summary, copolymers of
i) unsaturated carboxylic acids of the formula VII
   R¹(R²)C═C(R³)COOH (VII)
   in which R¹ to R³ are each independently —H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms,
ii) monomers of the formula VIII containing sulfonic acid groups
   R⁵(R⁶)C═C(R⁷)—X—SO₃H (VIII)
   in which R⁵ to R⁷ are each independently —H, —CH₃, a straight-chain or branched saturated alkyl radical having from 2 to 12 carbon atoms, a straight-chain or branched, mono- or polyunsaturated alkenyl radical having from 2 to 12 carbon atoms, alkyl or alkenyl radicals as defined above and substituted by —NH₂, —OH or —COOH, or are —COOH or —COOR⁴ where R⁴ is a saturated or unsaturated, straight-chain or branched hydrocarbon radical having from 1 to 12 carbon atoms, and X is an optionally present spacer group which is selected from —(CH₂)ₙ— where n=from 0 to 4, —COO—(CH₂)ₖ— where k=from 1 to 6, —C(O)—NH—C(CH₃)₂— and —C(O)—NH—CH(CH₂CH₃)—
iii) optionally further ionic or nonionogenic monomers are particularly preferred ingredients of the inventive laundry detergent and cleaning composition tablets.

Particularly preferred copolymers consist of
i) one or more unsaturated carboxylic acids from the group of acrylic acid, methacrylic acid and/or maleic acid,
ii) one or more monomers containing sulfonic acid groups of the formulae VIIIa, VIIIb and/or VIIIc:
   H₂C═CH—X—SO₃H (VIIIa)
   H₂C═C(CH₃)—X—SO₃H (VIIIb)
   HO₃S—X—(R⁶)C═C(R⁷)—X—SO₃H (VIIIc)
   in which R⁶ and R⁷ are each independently selected from —H, —CH₃, —CH₂CH₃, —CH₂CH₂CH₃, —CH(CH₃)₂ and X is an optionally present spacer group which is selected from —(CH₂)ₙ— where n=from 0 to 4, —COO—(CH₂)ₖ— where k=from 1 to 6, —C(O)—NH—C(CH₃)₂— and —C(O)—NH—CH(CH₂CH₃)—
iii) optionally further ionic or nonionogenic monomers.

The copolymers may contain the monomers from groups i) and ii) and optionally iii) in varying amounts, and it is possible to combine any of the representatives from group i) with any of the representatives from group ii) and any of the representatives from group iii). Particularly preferred polymers have certain structural units which are described below.

Thus, preference is given, for example, to inventive laundry detergent or cleaning composition tablets which are characterized in that they comprise one or more copolymers which contain structural units of the formula VIII
—[CH₂—CHCOOH]ₘ—[CH₂—CHC(O)—Y—SO₃H]ₚ— (VIII)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

These polymers are prepared by copolymerization of acrylic acid with an acrylic acid derivative containing sulfonic acid groups. Copolymerizing the acrylic acid derivative containing sulfonic acid groups with methacrylic acid leads to another polymer, the use of which in the inventive laundry detergent or cleaning composition tablets is likewise preferred and which is characterized in that the laundry detergent or cleaning composition tablets comprise one or more copolymers which contain structural units of the formula IX
—[CH₂—C(CH₃)COOH]ₘ—[CH₂—CHC(O)—Y—SO₃H]ₚ— (IX)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

Acrylic acid and/or methacrylic acid can also be copolymerized entirely analogously with methacrylic acid derivatives containing sulfonic acid groups, which changes the structural units within the molecule. Thus, inventive laundry detergent or cleaning composition tablets which comprise one or more copolymers which contain structural units of the formula X
—[CH₂—CHCOOH]ₘ—[CH₂—C(CH₃)C(O)—Y—SO₃H]ₚ— (X)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—, are likewise a preferred embodiment of the present invention, just like laundry detergent and cleaning composition tablets which are characterized in that they comprise one or more copolymers which contain structural units of the formula XI
—[CH₂—C(CH₃)COOH]ₘ—[CH₂—C(CH₃)C(O)—Y—SO₃H]ₚ— (XI)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

Instead of acrylic acid and/or methacrylic acid, or in addition thereto, it is also possible to use maleic acid as a particularly preferred monomer from group i). This leads to laundry detergent or cleaning composition tablets preferred in accordance with the invention which are characterized in that they comprise one or more copolymers which contain structural units of the formula XII
[HOOCCH—CHCOOH]ₘ—[CH₂—CHC(O)—Y—SO₃H]ₚ— (XII)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—, and to laundry detergent or cleaning composition tablets which are characterized in that they comprise one or more copolymers which contain structural units of the formula XIII
—[HOOCCH—CHCOOH]ₘ—[CH₂—C(CH₃)C(O)O—Y—SO₃H]ₚ— (XIII)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂C₃)—.

In summary, preference is given to laundry detergent or cleaning composition tablets according to the invention which comprise one or more copolymers which contain structural units of the formulae VIII and/or IX and/or X and/or XI and/or XII and/or XIII
—[CH₂—CHCOOH]ₘ—[CH₂—CHC(O)—Y—SO₃H]ₚ— (VIII)
—[CH₂—C(CH₃)COOH]ₘ—[CH₂—CHC(O)—Y—SO₃H]ₚ— (IX)
—[CH₂—CHCOOH]ₘ—[CH₂—C(CH₃)C(O)—Y—SO₃H]ₚ— (X)
—[CH₂—C(CH₃)COOH]ₘ—[CH₂—C(CH₃)C(O)—Y—SO₃H]ₚ— (XI)
—[HOOCCH—CHCOOH]ₘ—[CH₂—CHC(O)—Y—SO₃H]ₚ— (XII)
—[HOOCCH—CHCOOH]ₘ—[CH₂—C(CH₃)C(O)O—Y—SO₃H]ₚ— (XIII)
in which m and p are each a whole natural number between 1 and 2000, and Y is a spacer group which is selected from substituted or unsubstituted, aliphatic, aromatic or araliphatic hydrocarbon radicals having from 1 to 24 carbon atoms, preference being given to spacer groups in which Y is —O—(CH₂)ₙ— where n=from 0 to 4, is —O—(C₆H₄)—, is —NH—C(CH₃)₂— or —NH—CH(CH₂CH₃)—.

In the polymers, all or some of the sulfonic acid groups may be in neutralized form, i.e. the acidic hydrogen atom of the sulfonic acid group may be replaced in some or all of the sulfonic acid groups by metal ions, preferably alkali metal ions and in particular by sodium ions. Corresponding laundry detergent or cleaning composition tablets which are characterized in that the sulfonic acid groups within the copolymer are present in partially or completely neutralized form are preferred in accordance with the invention.

The monomer distribution of the copolymers used in the inventive laundry detergent or cleaning composition tablets is, in the case of copolymers which contain only monomers from groups i) and ii), preferably in each case from 5 to 95% by weight of i) or ii), more preferably from 50 to 90% by weight of monomer from group i) and from 10 to 50% by weight of monomer from group ii), based in each case on the polymer.

In the case of terpolymers, particular preference is given to those which contain from 20 to 85% by weight of monomer from group i), from 10 to 60% by weight of monomer from group ii), and from 5 to 30% by weight of monomer from group iii).

The molar mass of the sulfo copolymers described above and used in the inventive laundry detergent or cleaning composition tablets can be varied in order to adapt the properties of the polymers to the desired end use. Preferred laundry detergent or cleaning composition tablets are characterized in that the copolymers have molar masses of from 2000 to 200 000 gmol⁻¹, preferably from 4000 to 25 000 gmol⁻¹ and in particular from 5000 to 15 000 gmol⁻¹.

In addition to the substances from the substance classes mentioned, the inventive compositions may comprise further customary ingredients of detergents, of which bleaches, enzymes, silver protectants, dyes and fragrances are of particular significance. These substances are described below.

Among the compounds which serve as bleaches and supply H₂O₂ in water, sodium perborate tetrahydrate and sodium perborate monohydrate are of particular significance. Further bleaches which can be used are, for example, sodium percarbonate, peroxypyrophosphates, citrate perhydrates, and H₂O₂-supplying peracidic salts or peracids, such as perbenzoates, peroxophthalates, diperazelaic acid, phthaloimino peracid or diperdodecanedioic acid. Inventive detergents may also comprise bleaches, from the group of organic bleaches. Typical organic bleaches are the diacyl peroxides, for example dibenzoyl peroxide. Further typical organic bleaches are the peroxy acids, particular examples being the alkyl peroxy acids and the aryl peroxy acids. Preferred representatives are (a) the peroxybenzoic acid and ring-substituted derivatives thereof, such as alkylperoxybenzoic acids, but also peroxy-α-naphthoic acid and magnesium monoperphthalate, (b) the aliphatic or substituted aliphatic peroxy acids, such as peroxylauric acid, peroxystearic acid, ε-phthalimidoperoxycaproic acid [phthaloiminoperoxyhexanoic acid (PAP)], o-carboxybenzamidoperoxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-diperoxyazelaic acid, diperoxysebacic acid, diperoxybrassylic acid, the diperoxyphthalic acids, 2-decyldiperoxybutane-1,4-dioic acid and N,N-terephthaloyldi(6-aminopercaproic acid) may be used.

Bleaches used in the inventive laundry detergent or cleaning composition tablets may also be substances which release chlorine or bromine. Among suitable chlorine- or bromine-releasing materials, useful examples include heterocyclic N-bromoamides and N-chloroamides, for example trichloroisocyanuric acid, tribromoisocyanuric acid, dibromoisocyanuric acid and/or dichloroisocyanuric acid (DICA) and/or salts thereof with cations such as potassium and sodium. Hydantoin compounds, such as 1,3-dichloro-5,5-dimethylhydantoin, are likewise suitable.

Preferred inventive laundry detergent or cleaning composition tablets additionally contain bleaches in amounts of from 1 to 40% by weight, preferably from 2.5 to 30% by weight and in particular from 5 to 20% by weight, based in each case on the entire composition.

Bleach activators which may be used are compounds which, under perhydrolysis conditions, give rise to aliphatic peroxocarboxylic acids having preferably from 1 to 10 carbon atoms, in particular from 2 to 4 carbon atoms, and/or optionally substituted perbenzoic acid. Suitable substances bear O-acyl and/or N-acyl groups of the number of carbon atoms specified, and/or optionally substituted benzoyl groups. Preference is given to polyacylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic anhydrides, in particular phthalic anhydride, acylated polyhydric alcohols, in particular triacetin, ethylene glycol diacetate, 2,5-diacetoxy-2,5-dihydrofuran, n-methylmorpholiniumacetonitril methylsulfate (MMA), and also acetylated sorbitol and mannitol or mixtures thereof (SORMAN), acylated sugar derivatives, in particular pentaacetylglucose (PAG), pentaacetylfructose, tetraacetylxylose and octaacetyllactose, and acetylated, optionally N-alkylated, glucamine and gluconolactone, and/or N-acylated lactams, for example N-benzoylcaprolactam. Hydrophilically substituted acylacetals and acyllactams are likewise used with preference. Combinations of conventional bleach activators can also be used. The bleach activators are used in machine dishwasher detergents typically in amounts of from 0.1 to 20% by weight, preferably from 0.25 to 15% by weight and in particular from 1 to 10% by weight, based in each case on the composition. In the context of the present invention, the proportions specified are based on the weight of the base tablet.

In addition to the conventional bleach activators, or instead of them, it is also possible to incorporate bleach catalysts into the active substance particles. These substances are bleach-boosting transition metal salts or transition metal complexes, for example salen or carbonyl complexes of Mn, Fe, Co, Ru or Mo. It is also possible to use complexes of Mn, Fe, Co, Ru, Mo, Ti, V and Cu with N-containing tripod ligands, and also Co—, Fe—, Cu— and Ru-ammine complexes as bleach catalysts.

Preference is also given in accordance with the invention to using nitrile quats as bleach activators. Nitrile quats are cationic nitrites of the formula (XIV) 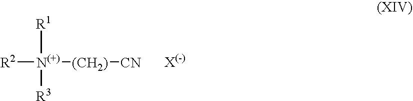
in which R¹ is —H, —CH₃, a C₂₋₂₄-alkyl or -alkenyl radical, a substituted C₂₋₂₄-alkyl or -alkenyl radical having at least one substituent from the group of —Cl, —Br, —OH, —NH₂, —CN, an alkyl- or alkenylaryl radical having a C₁₋₂₄-alkyl group, or is a substituted alkyl- or alkenylaryl radical having a C₁₋₂₄-alkyl group and at least one further substituent on the aromatic ring, R² and R³ are each independently selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂—CH₂—O)ₙH where n=1, 2, 3, 4, 5 or 6, and X is an anion.

The inventive tablets may comprise the cationic nitrites of the general formula (XIV) in varying amounts, the amount depending upon the end use of the tablets. For instance, laundry detergent tablets and cleaning composition tablets for machine dishwashing typically contain less bleach activator than, for example, bleach tablets which consist in large parts of bleach and bleach activator. Laundry detergent and cleaning composition tablets which are preferred in the context of the present invention are characterized in that they contain the cationic nitrile of the formula (XIV) in amounts of from 0.1 to 20% by weight, preferably from 0.25 to 15% by weight and in particular from 0.5 to 10% by weight, based in each case on the tablet weight.

The general formula (XIV) embraces a multitude of cationic nitriles which can be used in the context of the present invention. With particular advantage, the inventive laundry detergent and cleaning composition tablets comprise cationic nitriles in which R¹ is methyl, ethyl, propyl, isopropyl or an n-butyl, n-hexyl, n-octyl, n-decyl, n-dodecyl, n-tetradecyl, n-hexadecyl or n-octadecyl radical. R² and R³ are preferably selected from methyl, ethyl, propyl, isopropyl and hydroxyethyl, and one or both radicals may advantageously also be a cyanomethylene radical. In the table which follows, cationic nitriles of the formula (XIV) which are preferred in accordance with the invention are characterized by their R¹, R² and R³ radical:

| R¹ | R² | R³ |
|---|---|---|
| —H | —CH₃ | —CH₃ |
| —H | —CH₂—CH₃ | —CH₃ |
| —H | —CH₂—CH₂—CH₃ | —CH₃ |
| —H | —CH(CH₃)—CH₃ | —CH₃ |
| —H | —CH₂—OH | —CH₃ |
| —H | —CH₂—CH₂—OH | —CH₃ |
| —H | —CH(OH)—CH₃ | —CH₃ |
| —H | —CH₂—CH₂—CH₂—OH | —CH₃ |
| —H | —CH₂—CH(OH)—CH₃ | —CH₃ |
| —H | —CH(OH)—CH₂—CH₃ | —CH₃ |
| —H | —(CH₂CH₂—O)₁H | —CH₃ |
| —H | —(CH₂CH₂—O)₂H | —CH₃ |
| —H | —(CH₂CH₂—O)₃H | —CH₃ |
| —H | —(CH₂CH₂—O)₄H | —CH₃ |
| —H | —(CH₂CH₂—O)₅H | —CH₃ |
| —H | —(CH₂CH₂—O)₆H | —CH₃ |
| —CH₃ | —CH₃ | —CH₃ |
| —CH₃ | —CH₂—CH₃ | —CH₃ |
| —CH₃ | —CH₂—CH₂—CH₃ | —CH₃ |
| —CH₃ | —CH(CH₃)—CH₃ | —CH₃ |
| —CH₃ | —CH₂—OH | —CH₃ |
| —CH₃ | —CH₂—CH₂—OH | —CH₃ |
| —CH₃ | —CH(OH)—CH₃ | —CH₃ |
| —CH₃ | —CH₂—CH₂—CH₂—OH | —CH₃ |
| —CH₃ | —CH₂—CH(OH)—CH₃ | —CH₃ |
| —CH₃ | —CH(OH)—CH₂—CH₃ | —CH₃ |
| —CH₃ | —(CH₂CH₂—O)₁H | —CH₃ |
| —CH₃ | —(CH₂CH₂—O)₂H | —CH₃ |
| —CH₃ | —(CH₂CH₂—O)₃H | —CH₃ |
| —CH₃ | —(CH₂CH₂—O)₄H | —CH₃ |
| —CH₃ | —(CH₂CH₂—O)₅H | —CH₃ |
| —CH₃ | —(CH₂CH₂—O)₆H | —CH₃ |
| —CH₂—CH₃ | —CH₂—CH₃ | —CH₃ |
| —CH₂—CH₃ | —CH₂—CH₂—CH₃ | —CH₃ |
| —CH₂—CH₃ | —CH(CH₃)—CH₃ | —CH₃ |
| —CH₂—CH₃ | —CH₂—OH | —CH₃ |
| —CH₂—CH₃ | —CH₂—CH₂—OH | —CH₃ |
| —CH₂—CH₃ | —CH(OH)—CH₃ | —CH₃ |
| —CH₂—CH₃ | —CH₂—CH₂—CH₂—OH | —CH₃ |
| —CH₂—CH₃ | —CH₂—CH(OH)—CH₃ | —CH₃ |
| —CH₂—CH₃ | —CH(OH)—CH₂—CH₃ | —CH₃ |
| —CH₂—CH₃ | —CH₂—CH₃ | —CH₂—CH₃ |
| —CH₂—CH₃ | —CH₂—CH₂—CH₃ | —CH₂—CH₃ |
| —CH₂—CH₃ | —CH(CH₃)—CH₃ | —CH₂—CH₃ |
| —CH₂—CH₃ | —CH₂—OH | —CH₂—CH₃ |
| —CH₂—CH₃ | —CH₂—CH₂—OH | —CH₂—CH₃ |
| —CH₂—CH₃ | —CH(OH)—CH₃ | —CH₂—CH₃ |
| —CH₂—CH₃ | —CH₂—CH₂—CH₂—OH | —CH₂—CH₃ |
| —CH₂—CH₃ | —CH₂—CH(OH)—CH₃ | —CH₂—CH₃ |
| —CH₂—CH₃ | —CH(OH)—CH₂—CH₃ | —CH₂—CH₃ |
| —CH₂—CH₂—CH₃ | —CH₂—CH₂—CH₃ | —CH₃ |
| —CH₂—CH₂—CH₃ | —CH(CH₃)—CH₃ | —CH₃ |
| —CH₂—CH₂—CH₃ | —CH₂—OH | —CH₃ |
| —CH₂—CH₂—CH₃ | —CH₂—CH₂—OH | —CH₃ |
| —CH₂—CH₂—CH₃ | —CH(OH)—CH₃ | —CH₃ |
| —CH₂—CH₂—CH₃ | —CH₂—CH₂—CH₂—OH | —CH₃ |
| —CH₂—CH₂—CH₃ | —CH₂—CH(OH)—CH₃ | —CH₃ |
| —CH₂—CH₂—CH₃ | —CH(OH)—CH₂—CH₃ | —CH₃ |
| —CH₂—CH₂—CH₃ | —CH₂—CH₂—CH₃ | —CH₂—CH₃ |
| —CH₂—CH₂—CH₃ | —CH(CH₃)—CH₃ | —CH₂—CH₃ |
| —CH₂—CH₂—CH₃ | —CH₂—OH | —CH₂—CH₃ |
| —CH₂—CH₂—CH₃ | —CH₂—CH₂—OH | —CH₂—CH₃ |
| —CH₂—CH₂—CH₃ | —CH(OH)—CH₃ | —CH₂—CH₃ |
| —CH₂—CH₂—CH₃ | —CH₂—CH₂—CH₂—OH | —CH₂—CH₃ |
| —CH₂—CH₂—CH₃ | —CH₂—CH(OH)—CH₃ | —CH₂—CH₃ |
| —CH₂—CH₂—CH₃ | —CH(OH)—CH₂—CH₃ | —CH₂—CH₃ |
| —CH(CH₃)—CH₃ | —CH(CH₃)—CH₃ | —CH₃ |
| —CH(CH₃)—CH₃ | —CH₂—OH | —CH₃ |
| —CH(CH₃)—CH₃ | —CH₂—CH₂—OH | —CH₃ |
| —CH(CH₃)—CH₃ | —CH(OH)—CH₃ | —CH₃ |
| —CH(CH₃)—CH₃ | —CH₂—CH₂—CH₂—OH | —CH₃ |
| —CH(CH₃)—CH₃ | —CH₂—CH(OH)—CH₃ | —CH₃ |
| —CH(CH₃)—CH₃ | —CH(OH)—CH₂—CH₃ | —CH₃ |
| —CH(CH₃)—CH₃ | —CH₃ | —CH₂—CH₃ |
| —CH(CH₃)—CH₃ | —CH₂—CH₃ | —CH₂—CH₃ |
| —CH(CH₃)—CH₃ | —CH₂—CH₂—CH₃ | —CH₂—CH₃ |
| —CH(CH₃)—CH₃ | —CH(CH₃)—CH₃ | —CH₂—CH₃ |
| —CH(CH₃)—CH₃ | —CH₂—OH | —CH₂—CH₃ |
| —CH(CH₃)—CH₃ | —CH₂—CH₂—OH | —CH₂—CH₃ |
| —CH(CH₃)—CH₃ | —CH(OH)—CH₃ | —CH₂—CH₃ |
| —CH(CH₃)—CH₃ | —CH₂—CH₂—CH₂—OH | —CH₂—CH₃ |
| —CH(CH₃)—CH₃ | —CH₂—CH(OH)—CH₃ | —CH₂—CH₃ |
| —CH(CH₃)—CH₃ | —CH(OH)—CH₂—CH₃ | —CH₂—CH₃ |

For reasons of simple synthesis, preference is given to compounds in which the R¹ to R³ radicals are identical, for example (CH₃)₃N⁽⁺⁾CH₂—CN X⁻, (CH₃CH₂)₃N⁽⁺⁾CH₂—CN X^{-, (CH}₃CH₂CH₂)₃N ⁽⁺⁾CH₂—CN X⁻, (CH₃CH(CH₃))₃N(+)CH₂—CN X⁻, or (HO—CH₂—CH₂)₃N⁽⁺⁾CH₂—CN X⁻. Particular preference is given in accordance with the invention to one-phase or multiphase laundry detergent and cleaning composition tablets which comprise, as the cationic nitrile of the formula (XIV), a cationic nitrile of the formula (XIVa) 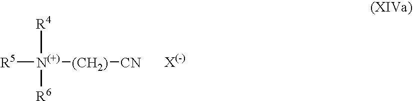
in which R⁴, R⁵ and R⁶ are each independently selected from —CH₃, —CH₂—CH₃, —CH₂—CH₂—CH₃, —CH(CH₃)—CH₃, where R⁴ may additionally also be —H, and X is an anion, preference being given to R⁵═R⁶═—CH₃ and in particular to R⁴═R═R⁶═—CH₃.

Particular preference is given in accordance with the invention to laundry detergent and cleaning composition tablets which comprise, as the cationic nitrile, (CH₃)₃N⁽⁺⁾CH₂—CN X⁻ where X⁻ is an anion which is selected from the group of chloride, bromide, iodide, hydrogensulfate, methosulfate, p-toluenesulfonate (tosylate) or xylenesulfonate.

When further bleach activators are used as well as the nitrile quats, preference is given to using bleach activators from the group of the polyacylated alkylenediamines, in particular tetraacetylethylenediamine (TAED), N-acylimides, in particular N-nonanoylsuccinimide (NOSI), acylated phenolsulfonates, in particular n-nonanoyl- or isononanoyloxybenzenesulfonate (n- or iso-NOBS), n-methylmorpholiniumacetonitrile methylsulfate (MMA), preferably in amounts up to 10% by weight, in particular from 0.1% by weight to 8% by weight, particularly from 2 to 8% by weight and more preferably from 2 to 6% by weight, based on the base tablet.

Bleach-boosting transition metal complexes, in particular with the central atoms Mn, Fe, Co, Cu, Mo, V, Ti and/or Ru, preferably selected from the group of manganese and/or cobalt salts and/or complexes, more preferably the cobalt (ammine) complexes, the cobalt (acetate) complexes, the cobalt (carbonyl) complexes, the chlorides of cobalt or manganese, and manganese sulfate, are used in customary amounts, preferably in an amount up to 5% by weight, in particular from 0.0025% by weight to 1% by weight and more preferably from 0.01% by weight to 0.25% by weight, based in each case on the overall composition. In specific cases, though, it is also possible to use a greater amount of bleach activator.

In order to ease the decomposition of highly compressed tablets, it is possible to incorporate disintegration assistants, known as tablet disintegrants, into the base tablet, in order to shorten disintegration times. Tablet disintegrants or disintegration accelerators refer to assistants according to Römpp (9th edition, vol. 6, p. 4440) and Voigt “Lehrbuch der pharmazeutischen Technologie” [Textbook of pharmaceutical technology] (6th edition, 1987, p. 182-184) which ensure the rapid decomposition of tablets in water or gastric juice and the release of pharmaceuticals in absorbable form.

These substances, which are also referred to as “breakup” agents owing to their action, increase their volume on ingress of water, and it is either the increase in the intrinsic volume (swelling) or the release of gases that can generate a pressure that causes the tablets to disintegrate into smaller particles. Disintegration assistants which have been known for some time are, for example, carbonate/citric acid systems, although other organic acids may also be used. Swelling disintegration assistants are, for example, synthetic polymers such as polyvinylpyrrolidone (PVP) or natural polymers or modified natural substances such as cellulose and starch and derivatives thereof, alginates or casein derivatives.

Preferred laundry detergent and cleaning composition tablets contain from 0.5 to 10% by weight, preferably from 3 to 7% by weight and in particular from 4 to 6% by weight, of one or more disintegration assistants, based in each case on the tablet weight. When only the base tablet comprises disintegration assistants, the values specified relate only to the weight of the base tablet.

Preferred disintegrants used in the context of the present invention are disintegrants based on cellulose, so that preferred laundry detergent and cleaning composition tablets contain such a cellulose-based disintegrant in amounts of from 0.5 to 10% by weight, preferably from 3 to 7% by weight and in particular from 4 to 6% by weight. Pure cellulose has the formal empirical composition (C₆H₁₀O₅)ₙ and, viewed in a formal sense, is a β-1,4-polyacetal of cellobiose which is in turn formed from two molecules of glucose. Suitable celluloses consist of from approx. 500 to 5000 glucose units and accordingly have average molar masses of from 50 000 to 500 000. Useful cellulose-based disintegrants in the context of the present invention are also cellulose derivatives which are obtainable by polymer-like reactions from cellulose.

Such chemically modified celluloses comprise, for example, products of esterifications and etherifications in which hydroxyl hydrogen atoms have been substituted. However, celluloses in which the hydroxyl groups have been replaced by functional groups which are not bonded via an oxygen atom can also be used as cellulose derivatives. The group of the cellulose derivatives includes, for example, alkali metal celluloses, carboxymethylcelluloses (CMC), cellulose esters and ethers, and amino celluloses. The cellulose derivatives mentioned are preferably not used alone as disintegrants based on cellulose, but rather in a mixture with cellulose. The content of cellulose derivatives in these mixtures is preferably below 50% by weight, more preferably below 20% by weight, based on the disintegrant based on cellulose. The disintegrant based on cellulose which is used is more preferably pure cellulose which is free of cellulose derivatives.

The cellulose used as a disintegration assistant is preferably not used in finely divided form, but rather converted to a coarser form before admixing with the premixtures to be compressed, for example granulated or compacted. The particle sizes of such disintegrants are usually above 200 μm, preferably to an extent of at least 90% by weight between 300 and 1600 μm and in particular to an extent of at least 90% by weight between 400 and 1200 μm. The aforementioned coarser cellulose-based disintegration assistants which are described in detail in the documents cited are to be used with preference as disintegration assistants in the context of the present invention and are commercially available, for example under the name Arbocel® TF-30-HG from Rettenmaier.

As a further cellulose-based disintegrant or as a constituent of this component, it is possible to use microcrystalline cellulose. This microcrystalline cellulose is obtained by partial hydrolysis of celluloses under conditions which attack and fully dissolve only the amorphous regions (approx. 30% of the total cellulose mass) of the celluloses, but leave the crystalline regions (approx. 70%) undamaged. A subsequent deaggregation of the microfine celluloses formed by the hydrolysis affords the microcrystalline celluloses which have primary particle sizes of approx. 5 μm and can be compacted, for example, to granules having an average particle size of 200 μm.

Laundry detergent and cleaning composition tablets which are preferred in the context of the present invention additionally contain a disintegration assistant, preferably a cellulose-based disintegration assistant, preferably in granulated, cogranulated or compacted form, in amounts of from 0.5 to 10% by weight, preferably from 3 to 7% by weight and in particular from 4 to 6% by weight, based in each case on the tablet weight.

The inventive laundry detergent and cleaning composition tablets may further comprise, either in the base tablet or in the cavity, a gas-evolving effervescent system. The gas-evolving effervescent system may consist of a single substance which releases a gas on contact with water. Among these compounds, mention should be made of magnesium peroxide in particular, which releases oxygen on contact with water. Typically, however, the gas-releasing effervescent system itself consists of at least two constituents which react with one another to form gas. While a multitude of systems which release, for example, nitrogen, oxygen or hydrogen are conceivable and practicable here, the effervescent system used in the inventive laundry detergent and cleaning composition tablets will be selectable on the basis of both economic and on the basis of environmental considerations. Preferred effervescent systems consist of alkali metal carbonate and/or alkali metal hydrogencarbonate and of an acidifier which is suitable for releasing carbon dioxide from the alkali metal salts in aqueous solution.

In the case of the alkali metal carbonates and/or alkali metal hydrogencarbonates, the sodium and potassium salts are distinctly preferred over the other salts for reasons of cost. It is of course not mandatory to use the pure alkali metal carbonates or alkali metal hydrogencarbonates in question; rather, mixtures of different carbonates and hydrogencarbonates may be preferred.

In preferred laundry detergent and cleaning composition tablets, the effervescent system used comprises from 2 to 20% by weight, preferably from 3 to 15% by weight and in particular from 5 to 10% by weight, of an alkali metal carbonate or alkali metal hydrogencarbonate, and from 1 to 15% by weight, preferably from 2 to 12% by weight and in particular from 3 to 10% by weight of an acidifier, based in each case on the overall tablet.

Acidifiers which release carbon dioxide from the alkali metal salts in aqueous solution and can be used are, for example, boric acid and also alkali metal hydrogensulfates, alkali metal dihydrogenphosphates and other inorganic salts. Preference is given, however, to the use of organic acidifiers, citric acid being a particularly preferred acidifier. However, it is also possible, in particular, to use the other solid mono-, oligo- and polycarboxylic acids. From this group, preference is given in turn to tartaric acid, succinic acid, malonic acid, adipic acid, maleic acid, fumaric acid, oxalic acid, and polyacrylic acid. It is likewise possible to use organic sulfonic acids such as amidosulfonic acid. A commercially available acidifier which can likewise be used with preference in the context of the present invention is Sokalan® DCS (trademark of BASF), a mixture of succinic acid (max. 31% by weight), glutaric acid (max. 50% by weight) and adipic acid (max. 33% by weight).

In the context of the present invention, preference is given to laundry detergent and cleaning composition tablets in which the acidifier used in the effervescent system is a substance from the group of the organic di-, tri- and oligocarboxylic acids, or mixtures of these.

In order to protect the ware or the machine, the inventive cleaning composition tablets may comprise, especially in the base tablet, corrosion inhibitors, and particularly silver protectants are of particular significance in the field of machine dishwashing. The known substances of the prior art can be used. Generally, it is possible in particular to use silver protectants selected from the group of the triazoles, the benzotriazoles, the bisbenzotriazoles, the aminotriazoles, the alkylaminotriazoles and the transition metal salts or complexes. Particular preference is given to using benzotriazble and/or alkylaminotriazole. Frequently also found in cleaning formulations are active chlorine-containing agents which can significantly reduce the corrosion of the silver surface. In chlorine-free detergents, particularly oxygen- and nitrogen-containing organic redox-active compounds, such as di- and trihydric phenols, for example hydroquinone, pyrocatechol, hydroxyhydroquinone, gallic acid, phloroglucine, pyrogallol and derivatives of these classes of compound are used. Salt- and complex-type inorganic compounds, such as salts of the metals Mn, Ti, Zr, Hf, V, Co and Ce, also frequently find use. Preference is given in this context to the transition metal salts which are selected from the group of manganese and/or cobalt salts and/or complexes, more preferably cobalt (ammine) complexes, cobalt (acetate) complexes, cobalt (carbonyl) complexes, the chlorides of cobalt or manganese, and manganese sulfate. Zinc compounds may likewise be used to prevent corrosion on the ware.

In addition to the builder, surfactant, disintegration assistant, bleach and bleach activator constituents mentioned, the inventive laundry detergent and cleaning composition tablets may comprise further ingredients, customary in laundry detergents and cleaning compositions, from the groups of the dyes, fragrances, optical brighteners, enzymes, foam inhibitors, silicone oils, antiredeposition agents, graying inhibitors, dye transfer inhibitors and corrosion inhibitors.

To increase the washing or cleaning performance, inventive compositions may contain enzymes, in which case it is possible in principle to use any enzymes established for these purposes in the prior art. These include in particular proteases, amylases, lipases, hemicellulases, cellulases or oxidoreductases, and preferably mixtures thereof. These enzymes are in principle of natural origin; starting from the natural molecules, improved variants are available for use in laundry detergents and cleaning compositions and are accordingly used with preference. Inventive compositions preferably contain enzymes in total amounts of from 1×10⁻⁶ to 5 percent by weight based on active protein. The protein concentration may be determined with the aid of known methods, for example the BCA method or the biuret method.

Among the proteases, preference is given to those of the subtilisin type. Examples thereof include the subtilisins BPN′ and Carlsberg, protease PB92, the subtilisins 147 and 309, Bacillus lentus alkaline protease, subtilisin DY and the enzymes thermitase and proteinase K which can be classified to the subtilases but no longer to the subtilisins in the narrower sense, and the proteases TW3 and TW7. The subtilisin Carlsberg is available in a developed form under the trade name Alcalase® from Novozymes A/S, Bagsvaerd, Denmark. The subtilisins 147 and 309 are sold under the trade names Esperase® and Savinase® respectively by Novozymes. The variants listed under the name BLAP® are derived from the protease of Bacillus lentus DSM 5483.

Further examples of useful proteases are the enzymes available under the trade names Durazym®, Relase®, Everlase®, Nafizym, Natalase®, Kannase® and Ovozymes® from Novozymes, those under the trade names Purafect®, Purafect®OxP and Properase® from Genencor, that under the trade name Protosol® from Advanced Biochemicals Ltd., Thane, India, that under the trade name Wuxi® from Wuxi Snyder Bioproducts Ltd., China, those under the trade names Proleather® and Protease P® from Amano Pharmaceuticals Ltd., Nagoya, Japan and that under the name Proteinase K-16 from Kao Corp., Tokyo, Japan.

Examples of amylases which can be used in accordance with the invention are the α-amylases from Bacillus licheniformis, from B. amyloliquefaciens or from B. stearothermophilus and developments thereof which have been improved for use in laundry detergents and cleaning compositions. The B. licheniformis enzyme is available from Novozymes under the name Termamyl® and from Genencor under the name Purastar®ST. Development products of this α-amylase are obtainable from Novozymes under the trade names Duramyl® and Termamyl®ultra, from Genencor under the name Purastar®OxAm and from Daiwa Seiko Inc., Tokyo, Japan as Keistase®. The B. amyloliquefaciens α-amylase is sold by Novozymes under the name BAN®, and variants derived from the B. stearothermophilus α-amylase under the names BSG® and Novamyl®, likewise from Novozymes.

Enzymes which should additionally be emphasized for this purpose are the α-amylase from Bacillus sp. A 7-7 (DSM 12368), and the cyclodextrin glucanotransferase (CGTase) from B. agaradherens (DSM 9948).

Also suitable are the developments of α-amylase from Aspergillus niger and A. oryzae, which are available under the trade names Fungamyl® from Novozymes. Another commercial product is Amylase-LT®, for example.

Inventive compositions may comprise lipases or cutinases, especially owing to their triglyceride-cleaving activities, but also in order to generate peracids in situ from suitable precursors. Examples thereof include the lipases which were originally obtainable from Humicola lanuginosa (Thermomyces lanuginosus) or have been developed, in particular those with the D96L amino acid substitution. They are sold, for example, under the trade names Lipolase®, Lipolase®Ultra, LipoPrime®, Lipozyme® and Lipex® from Novozymes. It is additionally possible, for example, to use the cutinases which have originally been isolated from Fusarium solani pisi and Humicola insolens. Lipases which are also useful can be obtained under the designations Lipase CE®, Lipase P®, Lipase B®, Lipase CES®, Lipase AKG®, Bacillis sp. Lipase®, Lipase AP®, Lipase M-AP® and Lipase AML® from Amano. Examples of lipases and cutinases from Genencor which can be used are those whose starting enzymes have originally been isolated from Pseudomonas mendocina and Fusarium solanii. Other important commercial products include the M1 Lipase® and Lipomax® preparations originally sold by Gist-Brocades and the enzymes sold under the names Lipase MY-30®, Lipase OF® and Lipase PL® by Meito Sangyo KK, Japan, and also the product Lumafast® from Genencor.

Inventive compositions may comprise further enzymes which are combined under the term hemicellulases. These include, for example, mannanases, xanthane lyases, pectin lyases (=pectinases), pectin esterases, pectate lyases, xyloglucanases (=xylanases), pullulanases and β-glucanases. Suitable mannanases are available, for example, under the names Gamanase® and Pektinex AR® from Novozymes, under the name Rohapec® B1L from AB Enzymes and under the name Pyrolase® from Diversa Corp., San Diego, Calif., USA. The β-glucanase obtained from B. subtilis is available under the name Cereflo® from Novozymes.

In order to enhance the bleaching action, inventive laundry detergents and cleaning compositions may comprise oxidoreductases, for example oxidases, oxygenases, catalases, peroxidases, such as haloperoxidases, chloroperoxidases, bromoperoxidases, lignin peroxidases, glucose peroxidases or manganese peroxidases, dioxygenases or laccases (phenol oxidases, polyphenol oxidases). Suitable commercial products include Denilite® 1 and 2 from Novozymes. Advantageously, preferably organic, more preferably aromatic, compounds which interact with the enzymes are additionally added in order to enhance the activity of the oxidoreductases concerned (enhancers), or to ensure the electron flux in the event of large differences in the redox potentials of the oxidizing enzymes and the soilings (mediators).

The enzymes used in inventive compositions either derive originally from microorganisms, for example of the genera Bacillus, Streptomyces, Humicola, or Pseudomonas, and/or are produced in biotechnology processes known per se by suitable microorganisms, for instance by transgenic expression hosts of the genera Bacillus or filamentous fungi.

The enzymes in question are favorably purified via processes which are established per se, for example via precipitation, sedimentation, concentration, filtration of the liquid phases, microfiltration, ultrafiltration, the action of chemicals, deodorization or suitable combinations of these steps.

The enzymes may be added to inventive compositions in any form established in the prior art. These include, for example, the solid preparations obtained by granulation, extrusion or lyophilization, or, especially in the case of liquid or gel-form compositions, solutions of the enzymes, advantageously highly concentrated, low in water and/or admixed with stabilizers.

Alternatively, the enzymes may be encapsulated either for the solid or for the liquid administration form, for example by spray-drying or extrusion of the enzyme solution together with a preferably natural polymer, or in the form of capsules, for example those in which the enzymes are enclosed as in a solidified gel, or in those of the core-shell type, in which an enzyme-containing core is coated with a water-, air- and/or chemical-impermeable protective layer. It is possible in layers applied thereto to additionally apply further active ingredients, for example stabilizers, emulsifiers, pigments, bleaches or dyes. Such capsules are applied by methods known per se, for example by agitated or roll granulation or in fluidized bed processes. Advantageously, such granules, for example as a result of application of polymeric film formers, are low-dusting and storage-stable owing to the coating.

It is also possible to formulate two or more enzymes together, so that a single granule has a plurality of enzyme activities.

A protein and/or enzyme present in an inventive composition may be protected, particularly during storage, from damage, for example inactivation, denaturation or decay, for instance by physical influences, oxidation or proteolytic cleavage. When the proteins and/or enzymes are obtained microbially, particular preference is given to inhibiting proteolysis, especially when the compositions also comprise proteases. For this purpose, inventive compositions may comprise stabilizers; the provision of such compositions constitutes a preferred embodiment of the present invention.

One group of stabilizers is that of reversible protease inhibitors. Frequently, benzamidine hydrochloride, borax, boric acids, boronic acids or salts or esters thereof are used, and of these in particular derivatives having aromatic groups, for example ortho-, meta- or para-substituted phenylboronic acids, or the salts or esters thereof. Peptidic protease inhibitors which should be mentioned include ovomucoid and leupeptin; an additional option is the formation of fusion proteins of proteases and peptide inhibitors.

Further enzyme stabilizers are amino alcohols such as mono-, di-, triethanol- and -propanolamine and mixtures thereof, aliphatic carboxylic acids up to C₁₂, such as succinic acid, other dicarboxylic acids or salts of the acids mentioned. Terminally capped fatty acid amide alkoxylates can also be used as stabilizers. Certain organic acids used as builders are additionally capable of stabilizing an enzyme which is present.

Lower aliphatic alcohols, but in particular polyols, for example glycerol, ethylene glycol, propylene glycol or sorbitol, are other frequently used enzyme stabilizers. Calcium salts are likewise used, for example calcium acetate or calcium formate, as are magnesium salts.

Polyamide oligomers or polymeric compounds such as lignin, water-soluble vinyl copolymers or cellulose ethers, acrylic polymers and/or polyamides stabilize the enzyme preparation against influences including physical influences or pH fluctuations. Polyamine N-oxide-containing polymers act as enzyme stabilizers. Other polymeric stabilizers are the linear C₈-C₁₈ polyoxyalkylenes. Alkylpolyglycosides can likewise stabilize the enzymatic components of the inventive composition and even increase their performance. Crosslinked N-containing compounds likewise act as enzyme stabilizers.

Reducing agents and antioxidants increase the stability of the enzymes against oxidative decay. An example of a sulfur-containing reducing agent is sodium sulfite.

Preference is given to using combinations of stabilizers, for example of polyols, boric acid and/or borax, the combination of boric acid or borate, reducing salts and succinic acid or other dicarboxylic acids or the combination of boric acid or borate with polyols or polyamino compounds and with reducing salts. The action of peptide-aldehyde stabilizers can be increased by the combination with boric acid and/or boric acid derivatives and polyols, and further enhanced by the additional use of divalent cations, for example calcium ions.

Preferred inventive machine dishwasher detergents are characterized in that they additionally comprise one or more enzymes and/or enzyme preparations, preferably solid and/or liquid protease preparations and/or amylase preparations, in amounts of from 1 to 5% by weight, preferably of from 1.5 to 4.5% by weight and in particular from 2 to 4% by weight, based in each case on the overall composition.

Dyes and fragrances may be added to the inventive machine dishwasher detergents in order to improve the esthetic impression of the resulting products and to provide the consumer with not only the performance, but also with a visually and sensorily “typical and unmistakable” product. The perfume oils and/or fragrances used may be individual odorant compounds, for example the synthetic products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methyl phenylglycinate, allyl cyclohexylpropionate, styrallyl propionate and benzyl salicylate. The ethers include, for example, benzyl ethyl ether; the aldehydes include, for example, the linear alkanals having 8-18 carbon atoms, citral, citronellal, citronellyloxyacetaldehyde, cyclamen aldehyde, hydroxycitronellal, lilial and bourgeonal; the ketones include, for example, the ionones, α-isomethylionone and methyl cedryl ketone; the alcohols include anethole, citronellol, eugenol, geraniol, linalool, phenylethyl alcohol and terpineol; the hydrocarbons include primarily the terpenes such as limonene and pinene. However, preference is given to using mixtures of different odorants which together produce a pleasing fragrance note. Such perfume oils may also comprise natural odorant mixtures, as are obtainable from vegetable sources, for example pine oil, citrus oil, jasmine oil, patchouli oil, rose oil or ylang-ylang oil. Likewise suitable are muscatel, sage oil, chamomile oil, clove oil, balm oil, mint oil, cinnamon leaf oil, lime blossom oil, juniper berry oil, vetiver oil, olibanum oil, galbanum oil and labdanum oil, and also orange blossom oil, neroli oil, orange peel oil and sandalwood oil.

The fragrances can be incorporated directly into the inventive cleaning compositions, but it may also be advantageous to apply the fragrances to carriers which ensure long-lasting fragrance by slower fragrance release. Useful such carrier materials have been found to be, for example, cyclodextrins, and the cyclodextrin-perfume complexes may additionally also be coated with further assistants.

In order to improve the esthetic impression of the inventive composition, it (or parts thereof) may be colored with suitable dyes. Preferred dyes, whose selection presents no difficulty at all to the person skilled in the art, have high storage stability and insensitivity toward the other ingredients of the compositions and to light, and also have no pronounced substantivity toward the substrates to be treated with the compositions, such as glass, ceramic or plastic dishes, so as not to stain them.

The inventive laundry detergent and cleaning composition tablets dissolve fully in the washing or cleaning cycle, and it can, as mentioned above, be advantageous for the different regions to have different dissolution rates. As a result of the different dissolution rates, it is possible not only for certain ingredients to be released at certain times but also for the properties of the washing or cleaning liquor to be modified in a controlled manner. For example, preference is given to laundry detergent and cleaning composition tablets for which the pH of a 1% by weight solution of the base tablet in water is in the range from 8 to 12, preferably from 9 to 11 and in particular from 9.5 to 10.

In addition, preference is given to laundry detergent and cleaning composition tablets for which the pH of a 1% by weight solution of the entire tablet in water is in the range from 7 to 11, preferably from 7.5 to 10 and in particular from 8 to 9.5.

The present invention further provides a process for producing filled depression tablets, which is characterized by the steps of
a) producing depression tablets;
b) introducing a solid depression filling into the depression;
c) fixing the depression filling with the aid of a fixative, a fixative-unfilled space being formed between the bottom of the depression in the base tablet and the depression filling.

With regard to the ingredients of the individual regions of the inventive tablet or of its particulate premixtures or compositions which give rise to the different regions of the tablet, the remarks made above for the inventive tablets apply analogously.

It has been found to be advantageous when the premixture compressed to base tablets in step a) satisfies certain physical criteria. Preferred processes are, for example, characterized in that the particulate premixture in step a) has a bulk density of at least 500 g/l, preferably at least 600 g/l and in particular at least 700 g/l.

The particle size of the premixture compressed in step a) also preferably satisfies certain criteria: preference is given in accordance with the invention to processes in which the particulate premixture in step a) has particle sizes between 100 and 2000 μm, preferably between 200 and 1800 μm, more preferably between 400 and 1600 μm and in particular between 600 and 1400 μm. To attain advantageous tablet properties, a particle size narrowed further in the premixtures to be compressed may be established. In preferred variants of the process according to the invention, the particulate premixture compressed in step a) has a particle size distribution in which less than 10% by weight, preferably less than 7.5% by weight and in particular less than 5% by weight, of the particles are larger than 1600 μm or smaller than 200 μm. Preference is further given here to narrower particle size distributions. Particularly advantageous process variants are characterized in that the particulate premixture compressed in step a) has a particle size distribution in which more than 30% by weight, preferably more than 40% by weight and in particular more than 50% by weight, of the particles have a particle size between 600 and 1000 μm.

In the performance of process step a), the process according to the invention is not restricted to the compression of only one particulate premixture to a tablet. Rather, process step a) can also be extended to the effect that multilayer tablets are produced in a manner known per se by preparing two or more premixtures which are compressed onto one another. In this case, the premixture introduced first is lightly precompressed in order to obtain a smooth upper side running parallel to the tablet bottom and, after the second premixture has been introduced, end-compressed to give the finished tablet. In the case of three-layer or multilayer tablets, a further precompression is effected after each premixture addition before end-compression is effected after addition of the last premixture of the tablets. The above-described cavity in the base tablet is preferably a depression, so that preferred embodiments of the first process according to the invention are characterized in that multilayer tablets which have a depression are produced in a manner known per se in step a) by pressing a plurality of different particulate premixtures onto one another.

The inventive tablets are produced in step a) initially by the dry mixing of the constituents which may be fully or partly pregranulated, and subsequently shaping, in particular compacting, to tablets, for which conventional processes can be employed. To produce the inventive tablets, the premixture is compacted in a die between two punches to form a solid compact. This operation, which is referred to below as tableting for short, divides into four sections: metering, compaction (elastic reshaping), plastic reshaping and expulsion.

First, the premixture is introduced into the die, the fill level and thus the weight and the shape of the resulting tablet being determined by the position of the lower punch and the shape of the compression tool. Even in the case of high tablet throughputs, the uniform metering is preferably achieved by volumetric metering of the premixture. In the further course of tableting, the upper punch contacts the premixture and descends further in the direction of the lower punch. In the course of this compaction, the particles of the premixture are pressed closer to one another, in the course of which the depression volume within the filling between the punches decreases continuously. From a certain position of the upper punch (and thus from a certain pressure on the premixture), plastic reshaping begins, in the course of which the particles coalesce and the tablet is formed. Depending on the physical properties of the premixture, a portion of the premixture particles is also crushed and there is sintering of the premixture at even higher pressures. At increasing compaction rate, i.e. high throughput amounts, the phase of elastic reshaping is shortened ever further, so that the resulting tablets can have cavities of greater or lesser size. In the last step of the tableting, the finished tablet is pushed out of the die by the lower punch and conveyed away by downstream transport devices. At this time, only the weight of the tablet has been ultimately defined, since the compacts may still change their shape and size owing to physical processes (elastic relaxation, crystallographic effects, cooling).

The tableting is effected in customary tableting presses which may in principle be equipped with single or double punches. In the latter case, not only the upper punch is used for pressure buildup; the lower punch also moves toward the upper punch during the compaction operation, while the upper punch presses downward. For small production amounts, preference is given to using eccentric tableting presses in which the punch(es) is/are secured to an eccentric disc which is in turn mounted on an axle having a particular rotation rate. The movement of these compression punches is comparable to the way in which a typical four-stroke engine works. The compaction can be effected with one upper and one lower punch, but a plurality of punches may also be secured to one eccentric disc, in which case the number of die bores is increased correspondingly. The throughputs of eccentric presses vary by type from a few hundred to a maximum of 3000 tablets per hour.

For greater throughputs, rotary tableting presses are selected, in which a greater number of dies is arranged in a circle on what is known as a die table. The number of dies varies by model between 6 and 55, larger dies also being commercially available. An upper and lower punch is assigned to each die on the die table, and the compression pressure can again be built up actively only by the upper or lower punch, or else by both punches. The die table and the punches move about a common vertical axis, the punches being brought into the positions for filling, compaction, plastic reshaping and expulsion with the aid of rail-like cam tracks during the rotation. At the points at which particularly severe raising or lowering of the punches is required (filling, compaction, expulsion), these cam tracks are supported by additional low-pressure sections, low-tension rails and discharge tracks. The dies are filled via a rigidly mounted feed apparatus, known as the filling shoe, which is connected to a stock vessel for the premixture. The compression pressure on the premixture can be adjusted individually via the compression paths for upper and lower punch, in which case the pressure is built up by virtue of the rolling movement of the punch shaft heads past adjustable pressure rolls.

To increase the throughput, rotary presses may also be provided with two filling shoes, in which case only one half-circle has to be passed through to produce one tablet. To produce two-layer and multilayer tablets, a plurality of filling shoes are arranged in series, without the lightly pressed first layer being expelled before the further filling. Suitable process control makes it possible in this way also to produce coated tablets and inlay tablets which have an onion-like structure, the top face of the core or of the core layers in the case of the inlay tablets not being covered and thus remaining visible. Rotary tableting presses can also be equipped with single or multiple tools, so that, for example, an outer circle having 50 bores and an inner circle having 35 bores may be utilized simultaneously for compression. The throughputs of modern rotary tableting presses are more than one million tablets per hour.

In the case of tableting with rotary presses, it has been found to be advantageous to carry out the tableting with minimum weight variations of the tablet. In this way, it is also possible to reduce the hardness variations of the tablet. Small weight variations can be achieved in the following manner:
use of plastic inlays having low thickness tolerances
low rotation rate of the rotor
large filling shoe
adjustment of the filling shoe vane rotation rate to the rotation rate of the rotor
filling shoe with constant powder height
decoupling of filling shoe and powder reservoir

To reduce caking on the punches, it is possible to use any antiadhesion coatings known from the art. Particularly advantageous antiadhesion coatings are plastic coatings, plastic inlays or plastic punches. Rotary punches have also been found to be advantageous, and upper and lower punch should be configured in a rotatable manner if possible. In the case of rotating punches, it is generally possible to dispense with a plastic inlay. In this case, the punch surfaces should be electropolished.

It has also been found that long pressing times are advantageous. These may be attained with pressure rails, a plurality of pressure rolls or low rotor rotation rates. Since the hardness variations of the tablet can be caused by the variations in the pressing forces, systems should be employed which restrict the pressing force. It is possible here to use elastic punches, pneumatic compensators or sprung elements in the force path. The pressure roll may also be of sprung design.

Processes preferred in the context of the present invention are characterized in that the compression in step a) is effected at compression pressures of from 0.01 to 50 kNcm⁻², preferably from 0.1 to 40 kNcm⁻² and in particular from 1 to 25 kNcm⁻².

Tableting machines suitable in the context of the present invention are, for example, obtainable from Apparatebau Holzwarth GbR, Asperg, Wilhelm Fette GmbH, Schwarzenbek, Hofer GmbH, Weil, Horn & Noack Pharmatechnik GmbH, Worms, IMA Verpackungssysteme GmbH Viersen, KILIAN Cologne, KOMAGE, Kell am See, KORSCH Pressen AG, Berlin, and Romaco GmbH, Worms. Further suppliers are, for example, Dr. Herbert Pete, Vienna (AU), Mapag Maschinenbau AG, Berne (CH), BWI Manesty, Liverpool (GB), I. Holand Ltd., Nottingham (GB), Courtoy N. V., Halle (BE/LU) and Mediopharm Kamnik (SI). A particularly suitable tableting press is, for example, the HPF 630 hydraulic double-pressure press from LAEIS, Germany. Tableting tools are available, for example, from Adams Tablettierwerkzeuge, Dresden, Wilhelm Fett GmbH, Schwarzenbek, Klaus Hammer, Solingen, Herber % Söhne GmbH, Hamburg, Hofer GmbH, Weil, Horn & Noack, Pharmatechnik GmbH, Worms, Ritter Pharmatechnik GmbH, Hamburg, Romaco, GmbH, Worms and Notter Werkzeugbau, Tamm. Further suppliers are, for example, Senss A G, Reinach (CH) and Medicopharm, Kamnik (SI).

In step b), a solid depression filling is introduced into the depression, and a contact surface is formed between the bottom of the depression in the base tablet and the depression filling. As has already been detailed above, preference is given here to using tablets as the depression filling, in which case the number of tablets to be introduced into the depression may be 1, 2, 3, 4, 5 or 6 and preference is given to inserting one tablet. Accordingly, preference is given to processes according to the invention in which the introduction of the solid depression filling in step b) comprises the placing of a tablet into the depression, the introduction being into the center of the depression.

Subsequently, the depression filling is fixed in the depression by the fixative in step c). The depression filling may be fixed via the depression walls, but the fixative may also cover the upper side of the depression filling or even the entire upper side of the tablet. As mentioned above, melts are preferred fixatives, so that preferred processes according to the invention are characterized in that the depression filling is fixed in step c) by pouring a melt of the fixative over it. In this context, it is possible and preferred that the fixative covers both the side surfaces of the depression filling and its upper surface.

The fixing of the depression filling may be supported by further measures. Especially in the case of the tablet-shaped depression filling, the shape and size of the depression may be selected such that the depression filling enters into a form fit or push fit with the depression bottom. Corresponding processes according to the invention in which the fixing of the tablet-shaped depression filling in step c) is supported by the geometry of tablet and depression bottom are accordingly preferred.

The process according to the invention can be carried out with high cycle rates without any risk of losses in quality. When the fixative is metered as a melt, it is possible to produce large amounts of depression tablets per unit time per metering station. Preference is given here to processes according to the invention in which the cycle rate of the fixative metering per metering station is ≧10 cycles/min, preferably ≧15 cycles/min and in particular ≧20 cycles/min.

1. A tablet comprising a base tablet having a depression and a solid filling the depression, wherein the solid filling the depression is secured in the depression by a fixative, and wherein a space is formed between the depression surface and the solid that is not filled with the fixative.

2. The tablet of claim 1, wherein a contact surface is formed between the depression surface in the base tablet and the fixative.

3. The tablet of claim 1, wherein the fixative fully covers the solid filling the depression.

4. The tablet of claim 1, wherein the surface of the depression and the solid filling the depression are separated by 0.5 to 10 mm.

5. The tablet of claim 4, wherein the surface of the depression and the solid filling the depression are separated by 1 to 9 mm.

6. The tablet of claim 5, wherein the surface of the depression and the solid filling the depression are separated by 2 to 8 mm.

7. The tablet of claim 6, wherein the surface of the depression and the solid filling the depression are separated by 3 to 7 mm.

8. The tablet of claim 1, wherein the solid filling the depression comprises a tablet having a density of ≧1 gcm⁻³.

9. The tablet of claim 8, wherein the solid filling the depression comprises a tablet having a density of ≧1.25 gcm⁻³.

10. The tablet of claim 9, wherein the solid filling the depression comprises a tablet having a density of ≧1.5 gcm⁻³.

11. The tablet of claim 1, wherein the fixative comprises one or more meltable substances that have a melting point between 30 and 250° C.

12. The tablet of claim 11, wherein the fixative comprises one or more meltable substances that have a melting point between 35 and 200° C.

13. The tablet of claim 12, wherein the fixative comprises one or more meltable substances that have a melting point between 40 and 180° C.

14. The tablet of claim 1, wherein the fixative comprises one or more substances selected from the group consisting of sugars, sugar acids, sugar alcohols, oligosaccharides, oligosaccharide derivatives, monosaccharides, disaccharides, monosaccharide derivatives, disaccharide derivatives, and any mixtures thereof.

15. The tablet of claim 14, wherein the fixative comprises glucose, fructose, ribose, maltose, lactose, sucrose, maltodextrin, Isomalt® or any mixture thereof.

16. A process for producing filled depression tablets, comprising the steps of
a) forming a base tablet having a depression;
b) introducing a solid depression filling into the depression in the base tablet;
c) fixing the depression filling with the aid of a fixative, a fixative-unfilled space being formed between the bottom of the depression in the base tablet and the depression filling.

17. The process of claim 8, wherein the introduction of the solid depression filling in step b) comprises the placing of a tablet into the depression, the introduction being into the center of the depression.

18. The process claim 8, wherein the depression filling is fixed in step c) by pouring a melt of the fixative over it.

19. The process of claim 18, wherein the fixative covers both the side surfaces of the depression filling and its upper surface.

20. The process of claim 16, wherein the fixing of the solid depression filling in step c) is supported by the configuration of the tablet and the depression surface.
